(19) United States
(12) Patent Application Publication
(10) Pub. No.: US 2022/0142214 A1
(43) Pub. Date: May 12, 2022

(54) SYNERGISTIC COMPOSITION OF FOOD-BASED AND ORGANIC NUTRIENTS AND METHODS FOR USE AND MANUFACTURE

(71) Applicant: StemSation USA, Inc., Plantation, FL (US)

(72) Inventor: Mira Gadzala, Plantation, FL (US)

(73) Assignee: StemSation USA, Inc., Plantation, FL (US)

(21) Appl. No.: 17/523,884

(22) Filed: Nov. 10, 2021

Related U.S. Application Data
(60) Provisional application No. 63/198,763, filed on Nov. 11, 2020.

(51) Int. Cl. A23L 7/10; A23L 29/244; A23L 33/115; A23L 33/135; A23L 33/21; A23L 33/00; A61K 31/593; A61K 36/31; A61K 36/074; A61K 36/9066; A61K 36/704; A61K 36/534; A61K 36/82

(52) U.S. Cl. CPC A23L 7/115; A23L 29/244; A23L 33/115; A23L 33/135; A23L 33/21; A23L 33/40; A61K 36/82; A61K 36/31; A61K 36/074; A61K 36/9066; A61K 36/704; A61K 36/534; A61K 31/593

(57) ABSTRACT

This application pertains generally to nutritional supplements comprising organic nutrients for consumption by a mammalian subject that supports and protects the immune system in a synergistic and complex way.

### RELATED APPLICATIONS

This application claims priority to provisional application No. U.S. Ser. No. 63/198,763 filed 11 Nov. 2020 which is hereby incorporated into this application in its entirety.

### FIELD OF THE DISCLOSURE

This application pertains generally to nutritional supplements comprising organic nutrients for consumption by a mammalian subject that supports and protects the immune system in a synergistic and complex way.

### BACKGROUND OF THE DISCLOSURE

This disclosure relates to nutritional supplements, and more particularly, to a novel synergistically acting composition of food-based, and organic nutrients providing a novel cellular nutrient delivery system, to effectively support and protect the immune system and the body's own ability to repair through optimal stem cell function and anti-aging mechanisms.

A compromised and imbalanced immune system leads to inflammation and chronic inflammation. Inflammatory processes are the underlying cause of almost all health problems and diseases, including autoimmune and cardiovascular diseases, cancer, diabetes, arthritis, and premature aging. Stress, unhealthy diet and lifestyle, exposure to toxins, smoking, intake of medication, unbalanced exercise, and age-related changes weaken the immune system. Over time, the immune system becomes less able to respond to all these challenges, and begins to decline. The current Coronavirus pandemic further contributes to health problems. There is also rapidly growing risk for other diseases in children and younger population.

Many supplements currently on the market contain random combinations of random compounds, often in mega-doses, that come from poor quality sources, and have never been tested. Many products currently on the market also contain synthetic, not natural, compounds often from genetically modified organism (GMO) sources, and contain unhealthy fillers, additives, or preservatives. Many products aim at supporting just isolated health aspects or processes in the body, leading to a lack of desirable health effects and more imbalances in the body function and nutritional deficiencies. A majority of supplements on the market do not support the body systems in a holistic and balanced way, and consequently do not provide expected health effects and improvements.

Further, most of dietary supplements aim at supporting isolated processes or health-related aspects, and do not support the immune system and the body function in a correct and balanced way. Many dietary supplements contain randomly combined and poor-quality compounds that often pollute rather than nourish cells, tissues, and body organs.

As can be seen, a solution is needed to these problems which may effectively support and protect the immune system and the body's own ability to repair for optimal function and health.

### SUMMARY OF THE DISCLOSURE

In some embodiments, this disclosure provides composition comprising an admixture of the nutraceutical components: beta-glucans from oats (Avena sativa); Astragalus extract; Broccoli sprouts extract (Brassica oleracea); Reishi mushroom extract (Ganoderma lucidum); Turmeric extract (Curcuma longa); 98% Trans-Resveratrol from Polygonum cupsidatum; Grape seed extract from Vitis rotundifolia; Apigenin from Green parsley extract (Petroselinum crispum), Green tea extract comprising 45%-50% EGCG (Epigallocatechin gallate, also known as epigallocatechin-3-gallate, is the ester of epigallocatechin and gallic acid, and is a type of catechin) from Camellia sinensis; Aloe vera leaf extract (BioAloe vera); Peppermint extract from Mentha piperita L.; and, Vitamin D3 (Cholecaliferol). Methods for making and using the compositions are also disclosed. Additional embodiments are also disclosed as will be understood by those of ordinary skill in the art.

### BRIEF DESCRIPTION OF THE DRAWINGS

FIG. 1. Growth and toxicity assay. Each panel is one representative image from three replicates used for measuring worm size and growth (C. elegans) with StemRCM exposure. Concentrations indicated above the top row were selected to span and highlight the transition from a non-toxic to a toxic dose. Images were acquired using WormLab imager and measured using automated detection and measurement.

FIG. 2. Growth and toxicity assay plots of worm size data obtained from high resolution imaging and automated detection and tracking software. Line=mean length or area, box=25th to 75^{th} percentile, whiskers=10th to 90th percentile.

FIG. 3. Acute toxicity assay. Large L4 stage worms were plated on solid media identical to that of the actual lifespan assay. Worms were examined for immediate toxic effects and then scored intermittently for survival until the start of Step 2 lifespan experiment. The 33.3 and 11.1 mg/mL, corresponding to media concentrations of 150 and 50 μg/mL in the media were selected for the longevity analysis.

FIG. 4A. Worm Activity (Aggregate Motility) analysis over duration of lifespan. Gross movement. Measured using Difference-Based Spatial Temporal Entropy Image (DSTEI)13

FIG. 4B. Worm Activity (Aggregate Motility) analysis over duration of lifespan. Average number of active worms per plate.

FIG. 5A. Morphology analysis over duration of lifespan. Length of worm is measured along a central spline fitted to the worm outline.

FIG. 5B. Morphology analysis over duration of lifespan. Width of worm is measured at the widest point orthogonal to the central spline.

FIG. 5C. Morphology analysis over duration of lifespan. Area is total pixel area of the worm outline converted to μm2.

FIG. 6. Average Circularity. Measures how close the worm's shape and posture is to a perfect circle, with a perfect circle having circularity of 1. Young, mobile worms have a slender shape and elongated posture, whereas aged/dying worms have a stouter shape and more curled posture.

FIG. 7. Connectivity of known aging-related pathways represented by genes differentially regulated under StemRCM treatment in young (Day 3) worms. Summary of pathway mapping to recognized aging-related pathways for StemRCM treatment in young (Day 3) worms. Colored score increments indicate the change of expression, up-(red) or down-(blue), weighted by the P value. Uncolored objects indicate components that were not detected in the data.

FIG. 8. Connectivity of known aging-related pathways represented by genes differentially regulated under StemRCM treatment aged (Day 10) worms. Summary of pathway mapping to established aging-related pathways for StemRCM treatment in aged (Day 10) worms. Colored score increments indicate the change of expression, up-(red) or down-(blue), weighted by the P value. Uncolored objects indicate components that were not detected in the data.

FIG. 9. Effects of novel combination of polyphenolic compounds and polysaccharides in StemRCM in reducing detrimental cellular effects of ROS versus a single compound—vitamin C.

### DETAILED DESCRIPTION

The following detailed description is of the best currently contemplated modes of carrying out exemplary embodiments of the invention. The description is not to be taken in a limiting sense but is made merely for the purpose of illustrating the general principles of this disclosure.

Broadly, an embodiment of this disclosure provides a novel synergistically-acting composition of food-based, the most potent, and organic nutrients—a combination of polyphenols and polysaccharides providing a novel cellular nutrient delivery system, to effectively support the detoxification pathways and processes, and protect the immune system and the body's own ability to repair through optimal stem cell function and anti-aging mechanisms.

The compositions of this disclosure, in an exemplary embodiment, may provide a blended model of innovative, synergistically acting, food-based, and the most potent, and organic nutrients—polyphenols and polysaccharides, providing a novel cellular nutrient delivery system, to effectively support and protect the detoxification pathways and processes, the immune system and the body's own ability to repair through optimal stem cell function and anti-aging mechanisms.

The compositions of this disclosure may provide a dietary-based solution to a compromised and imbalanced immune system, and the consequent inflammatory processes which are the underlying cause of almost all health problems and diseases, such as, for example without limitation, autoimmune and cardiovascular diseases, cancer, diabetes, arthritis, and premature aging. Stress, unhealthy diet and lifestyle, exposure to toxins, smoking, intake of medication, unbalanced exercise, and age-related changes weaken the immune system, and over time, it becomes less able to respond to all these challenges and begins to decline.

The compositions of this disclosure may provide superior, well-balanced, synergistic, and holistic nourishment for the immune system, cells, the body's stem cells repairing and healing activity, and other body systems and bio-cellular processes that work in synergy with the immune system or are required for its optimal function and health.

The compositions of this disclosure may comprise a dietary supplement which may provide novel and synergistic combination of all natural, and the most potent food-based compounds that work as natural immune booster and may help to significantly support and improve the immune system cell function, immune system responses to external disruptors, including viruses, bacteria, fungi, as well as internal disruptors (e.g. existing bio-cellular imbalances and inflammatory processes). The compositions of this disclosure may provide balanced, synergistic, and holistic nutritional support for optimal immune system function, other body systems that work in synergy with the immune system and help to improve the body's own stem cell repairing activity. All these aspects are critical for optimal health and wellness, prevention of health problems, premature aging, and health deterioration. The invention also embraces innovative nutrients absorption technology for optimal processing of the formula and significant health benefits.

In preferred embodiments, the compositions of this disclosure provide a synergistic and balanced combination of top-quality and all organic compounds—a novel combination of polyphenols and polysaccharides, from food sources that work as a team to assure the most optimal bio-cellular nutrition and health effects. The concept of synergy (or synergistic), as used in this disclosure, refers to the combined effect of the individual compounds to achieve much more significant health effects than application of individual compounds, or a random combination of compounds, or compounds provided in mega-doses, and allows to avoid mega-doses of compounds. There are numerous biochemical and metabolic pathways in the body and they are all connected. Nutrient synergy, unlike random combinations of random compounds, or isolated compounds, or compounds provided in a mega-dose, allows to support diverse biological targets at once and in a complex and balanced way, leading to optimal health effects. The invention also addresses the immune system function and health in a holistic way. The majority of supplements currently on the market contain random combinations of undefined nutrients, often in mega-doses, that come from poor quality sources, and aim at supporting just isolated health aspects or processes in the body leading to lack of desirable health effects and more imbalances in the body function.

The compositions of this disclosure provide an improvement over currently-available options. Most available dietary supplements aim at supporting isolated processes or health-related aspects, and do not support the immune system and the body function in a correct and balanced way. Such current supplements contain randomly combined and poor-quality compounds that often pollute rather than nourished cells, tissues, and body organs.

The compositions of this disclosure may provide a superior, most balanced, synergistic, and holistic nourishment for the immune system cell, stem cells, and other body systems and bio-cellular processes that work in synergy with the immune system or are required for its optimal function and health.

The compositions of this disclosure may also have multiple applications. The present compositions may be provided in a vegetarian capsule form, may also be tested and used as adjunct to conventional medical therapies. The present nutraceutical compositions may be formulated in different forms as well, such as powder or liquids, or can be even applied in a form of an injections (e.g. intramuscular) for immediate bio-cellular distribution, optimal nourishment, and health effects.

The Stem RCM formula (“present nutraceutical compostions”) contains novel and synergistic combination of the most potent, fast acting, and easily absorbed polyphenolic and polysaccharides extracts that come from organic plants, fruits, and food sources. The importance of researching the polyphenols-polysaccharides combination of natural compounds for the immune defense processes, and other processes, has been recently reported in the peer review journal—Scientific Reports (January 2021). The research of polyphenols-polysaccharides combination is gaining attention but the application of such combination to natural products is still uncommon in the nutraceutical market, making the StemRCM formula advanced and unique. Plant polyphenols are considered to be one of the most biologically active natural ingredients and potent antioxidants for the prevention and management of health problems and diseases due to their significant antioxidant and anti-inflammatory potential. Polyphenols modulate inflammatory response by regulating pro-inflammatory cytokines synthesis, immune system cells, stem cells, and gene regulation. However, the primary mechanism of polyphenols for immune-modulation is their multiple antioxidant capability, including lowering deleterious effects of cellular reactive oxygen species (ROS) level and free radicals that contribute to premature death of immune cells. The primary functions of antioxidants include the regulation of the redox potential within a cell and the reduction of potential initiators of cell death and carcinogenesis. Redox changes within a cell are able to trigger various molecular responses such as induction of apoptosis (cell death) and activation of signal transduction (the transfer of messages between cells and within a cell). Hence antioxidants are considered anti-carcinogenic and anti-inflammatory agents, and play an important role in protecting the immune system. For example, and independent study shows that the intraperitoneal administration of quercetin, apigenin, Epigallocathechin-3-gallate (EGCG), resveratrol, all present in StemRCM, and the anti-estrogen tamoxifen, at the time of intramuscular (i.m.) injection of B16-BL6 cells into syngeneic mice, resulted in a significant, dose-dependent delay of tumor growth, without toxicity. Furthermore, these polyphenolic compounds significantly potentiated the inhibitory effect of a non-toxic dose of cisplatin. When tested for the ability to inhibit lung colonization, quercetin, apigenin, and tamoxifen significantly decreased the number of B16-BL6 colonies in the lungs in a dose-dependent manner, with quercetin and apigenin being more effective than tamoxifen. In the study conclusion: “Quercetin and apigenin (both present in StemRCM) showed inhibitory effects on melanoma growth, and invasive and metastatic potential; therefore, they may constitute a valuable tool in the combination therapy of metastatic melanoma” (Int J Cancer. 2000 Aug. 15; 87(4):595-600). In the cited study, quercetin comes from a single and random source. In StemRCM, quercetin comes from multiple natural, organic, and highly potent sources, such as Muscadine grapes, Japanese knotweed root, green parsley, Broccoli sprouts, fast-absorbed Aloe vera, and green tea. The synergistic interaction of quercetin from these multiple natural and highly potent sources may accelerate its immune, anti-carcinogenic, and other health effects in the body and increase the synergistic immune and other health effects with apigenin.

Another group of innovative and potent immuno-modulating compounds, in StemRCM, are polysaccharides known as β-glucans, and they may work in synergy with polyphenols. Polysaccharides, similar to polyphenols, come in Stem RCM from multiple, highly potent, and fast acting natural sources such as organic oats, Reishi mushrooms, astragalus, Broccoli sprouts, and aloe vera (BioAloe). Numerous studies show that β-glucans have been implicated in the initiation of anti-microbial immune response. Based on in vitro studies, β-glucans act on several immune receptors including Dectin-1, complement receptor (CR3) and TLR-2/6, and trigger a group of immune cells including macrophages, neutrophils, monocytes, natural killer cells, and dendritic cells. As a consequence, β-glucans modulate both innate and adaptive responses, and can also enhance opsonic and non-opsonic phagocytosis. Polysaccharides activate the protein pathways, which in turn are activated by mitogens (MAPKs) and others such as the nuclear factor (NF-kB), stimulating the immune response control processes. Polysaccharides obtained from natural food sources also stimulate the production and expression of messenger RNA (mRNA) during the synthesis of nitric oxide and pro-inflammatory cytokine, and can significantly increase the expression of messenger RNA (mRNA) and cytokines by activating regulation of receptors such as TLR2 and TLR4. Most β-glucans, especially fast-acting like those present in StemRCM, enter the proximal small intestine and some are captured by the macrophages. They are internalized and fragmented within the cells, then transported by the macrophages to the bone marrow and endothelial reticular system. The small β-glucans fragments are eventually released by the macrophages and taken up by other immune cells leading to various immune responses. Careful selection of appropriate, fast-acting and quickly absorbed β-glucans is essential for their health benefits and further clinical investigation. Since β-glucans are inexpensive and have good margin of safety based on historical track records, their potential therapeutic value deserves further investigation in StemRCM formula.

The polyphenolic-polysaccharides combination of compounds started gaining research attention due to their health benefits, especially immune responses. In plant-based food systems such as fruits, vegetables, mushrooms, and cereals (all present in StemRCM), cell wall polysaccharides and polyphenols co-exist and commonly interact during processing in the body and digestion. Polysaccharides could attract phenolic compounds naturally via noncovalent binding such as hydrophobic interactions and hydrogen bonds, and may greatly influence their physicochemical, nutritional, and health properties. Oral administration of the combination of glucan and polyphenolic compounds offers significant advantages such as easy administration, demonstrated their therapeutic efficacy.

In preferred embodiments, the synergistic combination of polyphenols-polysaccharides in StemRCM can exhibit detoxification effects, preventive anti-inflammatory and tumor inhibitory effects, and stimulate immune stem cells. StemRCM can also provide repairing and healing effects when combined with conventional immune- and/or anticancer therapies.

In preferred embodiments, the compositions of this disclosure (e.g., StemRCM) may comprise one or more of the following elements, compounds or components and combinations thereof, as shown below:

1. Beta-glucans (from organic oats). Beta-glucans are polysaccharides, soluble fibers, and extremely potent, immune activating molecules. They strongly activate macrophages in the immune system that are the first line of defense against viral, bacterial and other infections. They have been documented to play a significant role against coronavirus infection. Macrophages are also essential for recognizing and eliminating aberrant and carcinogenic cells from the body. Beta glucans health benefits, including their ability to activate the body's stem cell healing and repairing function, have been documented and recognized in numerous research studies;

2. Apigenin from organic green parsley is a naturally occurring and highly potent plant flavonoid (polyphenolic compound) that has been documented in research studies to provide natural powerful anti-oxidant, anti-inflammatory, and anti-carcinogenic properties, and protection of DNA;

3. Organic Broccoli sprouts extract (35 mg sulforaphane) that is strong immune system booster and protector, supports detoxification pathways, and helps to detoxify from cancer causing toxins. Sulforaphane boosts Type 1 interferon responses to viruses, including coronavirus. Broccoli sprout extract in the compositions of this disclosure is additionally enriched in natural Myrosinase enzyme that is required for optimal digestion and assimilation of Broccoli. Many products on the market do not contain this important enzyme;

4. EGCG from organic green tea (45%-50% EGCG). This highly potent green tea extract is standardized to 85-95% polyphenols and EGCG is highly potent antioxidant and immune-modulator;

5. Organic Reishi mushroom extract, which have a long history of use in promoting vibrant health and longevity in China, Japan, and other Asian countries, is called “mushroom of immortality”, and is powerful antioxidant and provides natural immune-modulating effects and anti-carcinogenic protection;

6. Organic Trans-resveratrol (98% trans-resveratrol) from Japanese Knotweed root has the highest concentration of resveratrol among all plants. Resveratrol has been extensively research for its strong antioxidant, anti-inflammatory, and anti-aging and longevity effects, cardiac protection (French paradox), and enhancement of the immune system and energy endurance;

7. Organic Muscadine grape seed extract is a natural source of powerful antioxidants that has been documented in research studies to provide potent and natural anti-inflammatory, cardio-protective, and anti-aging effects, and support for the body's own stem cells healing and repairing activity. Muscadine grapes are called “health superstars” because they have 40× higher antioxidant level than regular grapes, and approximately 6× the resveratrol content versus regular grapes;

8. Organic curcumin, that has been developed based on Japanese nanotechnology that reduces curcumin molecules size 100 times and because of that, makes curcumin one of the most bioavailable curcumins on the market, and 27 times faster absorbed and assimilated, and quickly and effectively delivered to cellular targets than any other curcumins. Curcumin addresses inflammatory processes by naturally lowering or inhibiting levels of inflammatory markers, including C-Reactive Proteins (CRP), IL-6, other inflammatory cytokines and enzymes;

9. Organic Astragalus is known for its deep immune supporting properties, enhancing the body's own defense system while controlling autoimmune imbalances, promoting optimal levels of specific immune cells, especially T cells;

10. Vitamin D3 (as Cholecalciferol) plays a number of critical roles in protecting the human body and health, including the immune system support and activation against external and internal body invaders;

11. Organic Peppermint extract is a potent source of strong antioxidants, and provides a broad range of anti-bacterial, anti-inflammatory, anti-allergic, and calming effects, and also supports and relaxes the digestive system and helps to increase optimal processing of nutrients; and helps to increase optimal processing of nutrients;

12. Organic BioAloe is highly bioavailable (approximately 10× more bioavailable versus any regular Aloe vera extract) and quickly absorbed. It comes from the inner leaf, has the highest concentration of powerful immunomodulator—accemannan (<400 KDa), and is a naturally occurring polysaccharide documented in numerous studies to support the immune system function and cellular processes, which are responsible for the body's natural defense.

In preferred embodiments, the compositions of this disclosure comprise about 110-150 mg beta-glucans (from oats; preferably about 130 mg); about 80-120 mg Astragalus (Atragalus membranaceus; preferably 100 mg); about 80-120 mg broccoli sprouts extract (Brssica olercea; preferably about 100 mg); about 70-90 mg Reishi Mushroom (Ganoderma lucidum; preferably about 80 mg); about 40-60 mg curcumin (Theracurmin®; preferably about 50 mg); about 30-50 mg 98% trans-resveratrol (Polygonum cupsidtum; preferably about 40 mg); about 30-50 mg grape seed extract (Vitis rotundifolia; preferably about 40 mg); about 30-50 mg apigenin (green parsley extract; Petroselinum cripsum; preferably about 40 mg); about 20-40 mg green tea extract (EGCG; Camellia sinensis; preferably about 30 mg); about 10-20 mg Aloe vera (preferably about 15 mg); about 200 International Units (IU) (or about 5 micrograms) vitamin D3 (as Cholecalciferol); and, about 5-15 mg peppermint (Mentha piperita L.; preferably about 10 mg). Preferably, these components are comprised within a pharmaceutically acceptable capsule, preferably a vegetarian capsule. In some embodiments, each capsule comprises 500-700 mg, preferably about 640 mg, of these components. In some embodiments, the dosage is three capsules such that each dose comprises 1500-2100, preferably about 1900 mg of these components.

Without limitation and in an exemplary embodiment, the compositions of this disclosure may include the following properties and relationship between the components. The listed all-natural and organic compounds may help to increase each other's anti-oxidant and natural immunomodulatory responses and effects through various bio-cellular and genetic pathways. For example: apigenin (#2) when combined with Broccoli sprouts extract (#3) increases anti-oxidative and anti-inflammatory processes within the body, and their synergistic interaction also increases the induction of UGT1A1 enzyme that helps with detoxification processes. Apigenin (#2) when combined with EGCG (#4) and resveratrol (#6) helps to decrease activation of inflammatory processes and increase detoxification processes. The combination of Broccoli extract (#3) with Reishi mushrooms (#5) can result in increased immune system responses and function, and increased gene expression of NAD(P)H:quinone oxidoreductase. The synergistic interaction between EGCG (#4) and curcumin (#8) can help to activate the immune system responses and reduce cellular cytotoxicity.

The innovative combination of all listed compounds that come from two and synergistically combined groups: polyphenols and polysaccharides, and innovative addition of peppermint extract and apigenin, may provide significantly increased immune system responses, and immune enhancing benefits, and unveil new bio-cellular mechanisms that may be essential for strong immune system function, prevention of health problems and diseases, and healthy longevity.

The individual components (e.g., natural compounds) of the compositions of this disclosure have been individually documented in previous studies to be effective and safe in supporting certain immune system and anti-oxidative processes, and the body's natural stem cell physiology, meaning their natural release, circulation and migration to the location that requires repair. The individual components (ingredients) present in compositions of this disclosure have been also scientifically documented to provide DNA protection and support gene expression (among other). However, as individual components, they have limited activity, and are unable to address in a balanced and comprehensive way the multiple pathways and processes involved in supporting the immune system and other body system functions, stem cell physiology, and overall health. An intake of dietary supplements composed of one, two, or three compounds may support some processes in the body but only to some extent, and may, at the same time, trigger imbalances in other processes and body systems, and increase nutritional deficiency in cells and tissues. A side example is vitamin B12. When taken as individual nutrient, vitamin B12 can trigger an imbalance in folic acid level and a few other nutrients in the body, leading to nutritional deficiencies rather than preventing them. Also, taken as an individual compound, vitamin B12 is not well processed and assimilated.

Emerging studies document significantly increased health effects of certain nutrients combinations versus individual compounds. This is a new trend in science and medicine. The nutrient synergy concept and novel combination of polyphenolic compounds and polysaccharides of this disclosure, as well as the holistic aim of this disclosure at multiple pathways by a team of top-quality and fast-acting compounds, may provide significant and multiple health effects, long-lasting protection, and improvements. It may also unveil new and significant bio-cellular mechanism of actions and stem cell mobilization, and may provide new insight for addressing and improving immune system health and overall health in the most optimal and balanced way. The synergistic combination of compounds in the proposed innovation also helps to avoid mega-doses of single compounds and consequent bio-cellular imbalances. The novel composition provides advanced absorption technology of curcumin, which significantly increases its efficacy, assimilation, absorption, and fast delivery to cells. The organic curcumin present in the novel composition of this disclosure may be 27 times more bioavailable (faster absorbed and assimilated) than any other curcumin on the market. This disclosure may provide a dietary supplement capable of helping to protect, support, and improve the immune system function and human health in an unmatched way.

The compositions of this disclosure may be made by an innovative process requiring meticulous research, scientific investigations, and analysis. The compositions of this disclosure can be categorized in a dietary supplement category, may entail several steps and require deep knowledge in several fields, such as biomolecular, cellular, genetics, and orthomolecular field, botany, and holistic nutrition. The compositions of this disclosure require thorough analysis of dozens or even hundreds of existing research studies and clinical studies that document the efficacy and safety of natural compounds, especially those used in the invention. Laboratory and clinical testing of invention for efficacy, safety, and toxicity is a critical aspect, as well as knowledge and adherence to regulations of the components used in invention. Analysis of market reviews and trends in a category of invention is also an integral part of the invention process.

In the dietary supplement category, clinical studies with dietary supplements are optional and are not required. Dietary supplements are classified as generally recognized as safe (GRAS). Conducting a clinical study with this disclosure may show and document new mechanism of natural and novel nutrient combination, and initiate new directions in the immune system health and its protection.

The components of the composition are capable of working synergistically as a team, and have been provided in efficacious and balanced doses and ratio. The synergistic combination of the blend of this disclosure provides a unique, top-quality, composition. The removal of any component or its replacement by another, or the same but from poor quality or contaminated source may alter or significantly alter the potency and even safety of the invention. For example, a replacement of the highly bioavailable turmeric in compositions of this disclosure by any other regular turmeric, that is present in so many products, may decrease efficacy, assimilation, and processing of this compound and also other compounds in the compositions of this disclosure.

In an exemplary embodiment, the compositions of this disclosure may be used in the following manner to provide a novel combination of highly potent food-based compounds, a team of nutrients that come from organic sources, which may support, protect, and may help to improve the immune system function in a complex, balanced, and holistic way. By taking the composition of this disclosure, a user may observe and experience not only an improvement in the immune system function, but also other body function and overall health. The supplement composition of this disclosure may serve as a highly potent, safe, balanced, and innovative nutritional foundation for the immune system and other body systems and processes that are required for optimal immune system function. By taking the supplement composition of this disclosure, a person may also avoid taking multiple products in unhealthy mega-doses, save money, and may be able to nourish cells, tissues, and the body in the most beneficial way and achieve long-lasting health and anti-aging effects.

Also, compositions of this disclosure may be provided in multiple types of products in multiple forms. In some embodiments, compositions of this disclosure may be provided in a vegetarian capsule form, and may further be tested and used as adjuncts to conventional medical therapies. Compositions of this disclosure may be formulated in different forms as well, such as powders or liquids, or may even be administered or applied in a form of a vaccine (such as, for example without limitation, intramuscular injection) for immediate bio-cellular distribution, optimal nourishment, and health effects.

In summary, in an exemplary embodiment, the compositions of this disclosure may provide a team model of innovative, synergistically acting, food-based, and organic nutrients in combination, and a novel cellular nutrient delivery system, to effectively support and protect the immune system and the body's own ability to repair through optimal stem cells function and anti-aging mechanisms. The compositions of this disclosure may provide a superior, most balanced, synergistic, and holistic nourishment for the immune system cell, stem cells, and other body systems and bio-cellular processes that work in synergy with the immune system or are required for its optimal function and health.

Preferred embodiments, or preferred aspects, of this disclosure include but are not limited to the following. In some preferred embodiments, this disclosure provides compositions comprising an admixture of the nutraceutical components beta-glucans from oats; Astragalus extract; Brassica oleracea extract; Ganoderma lucidum (Reishi Mushroom) extract; Curcuma longa extract; Trans-Resveratrol from Polygonum cupsidatum; grape seed extract from Vitis rotundifolia; Apigenin from Green parsley extract (Petroselinum crispum), green tea extract comprising EGCG from Camellia sinensis; Aloe vera extract; peppermint extract from Mentha piperita L; and, Vitamin D3 (Cholecaliferol). In some preferred embodiments, this disclosure provides compositions comprising 110-150 mg of beta-glucans from oats; about 80-120 mg of Astragalus extract; about 80-120 mg of Brassica oleracea extract; about 70-90 mg of Ganoderma lucidum (Reishi Mushroom) extract; about 40-60 mg of Curcuma longa extract; about 30-50 mg of Trans-Resveratrol from Polygonum cupsidatum; about 30-50 mg of grape seed extract from Vitis rotundifolia; about 30-50 mg of apigenin from Green parsley extract (Petroselinum crispum), about 20-40 mg of green tea extract comprising EGCG from Camellia sinensis; about 10-20 mg of Aloe vera extract; about 5-15 mg peppermint extract from Mentha piperita L; and, about 150-250 IU or 1-5 microgram of Vitamin D3 (Cholecaliferol). In some preferred embodiments, this disclosure provides compositions comprising about 117-143 mg of the beta-glucans; about 80-120 mg of the Astragalus extract; about 90-110 mg of the Brassica oleracea extract; about 80 mg of the Ganoderma lucidum (Reishi Mushroom) extract; about 45-55 mg of the Curcuma longa extract; about 36-44 mg of the Trans-Resveratrol; about 36-44 mg of the grape seed extract; about 36-44 mg of the green parsley extract; about 27-33 mg of the green tea extract; about 13.5-16.5 mg of the Aloe vera extract; about 9-11 mg of the peppermint extract; and, about 1.8-3.0 micrograms of the vitamin D3 (Cholecaliferol). In some preferred embodiments, this disclosure provides compositions comprising about 130 mg of the beta-glucans; about 100 mg of the Astragalus extract; about 100 mg of the Brassica oleracea extract; about 80 mg of the Ganoderma lucidum (Reishi Mushroom) extract; about 50 mg of the Curcuma longa extract; about 40 mg of the Trans-Resveratrol; about 40 mg of the grape seed extract; about 40 mg of the green parsley extract; about 30 mg of the green tea extract; about 15 mg of the Aloe vera extract; about 10 mg of the peppermint extract; and, about 2 micrograms or 200 IU of the vitamin D3 (Cholecaliferol). In some preferred embodiments, this disclosure provides such compositions that further comprising a pharmaceutically acceptable excipient, optionally suitable for intravenous (IV) administration or contained in a capsule (e.g., a vegetarian capsule). In some preferred embodiments, this disclosure provides a capsule comprising a total of 500-700 mg, optionally about 640 mg, of the nutraceutical components. In some preferred embodiments, this disclosure provides dosage forms comprising one or more capsules comprising 1500-2100 mg, optionally about 1900 mg, of the nutraceutical components. In some preferred embodiments, this disclosure provides methods for altering the expression of one or more genes in C. elegans, the one or more genes selected from the group consisting of a gene in the C. elegans Insulin Signaling Pathway, mtl-1, sod-2/3, gst-4, gcs-1, cpr-1, daf-18, and daf-2; the method comprising administering a composition or dosage form of any preceding claim to C. elegans. In some preferred embodiments, this disclosure methods for altering the expression of one or more genes in a mammal, the one or more genes being a mammalian homologue selected from the group consisting of a gene in the C. elegans Insulin Signaling Pathway, mtl-1, sod-2/3, gst-4, gcs-1, cpr-1, daf-18, and daf-2; the method comprising administering a composition or dosage form of any preceding claim to a mammal. In some preferred embodiments, this disclosure provides compositions for use as a dietary supplement. In some preferred embodiments, this disclosure provides compositions that support anti-aging in vivo mechanisms. In some preferred embodiments, this disclosure provides compositions that supplements the immune system and/or reduces in vivo inflammation. In some preferred embodiments, this disclosure provides compositions that improves the bioavailability for each nutraceutical component thereof as compared to effects of each nutraceutical administered alone. Other embodiments are also provided by this disclosure as would be understood by those of ordinary skill in the art.

For clarity, only those aspects of the system germane to the invention are described, and product details well known in the art are omitted. In addition, many embodiments of this disclosure have application to a wide range of industries. To the extent the present application discloses a system, the method implemented by that system is within the scope of this disclosure. Further, to the extent the present application discloses a method, a system of apparatuses configured to implement the method are within the scope of this disclosure.

It should be understood, of course, that the foregoing relates to exemplary embodiments of the invention and that modifications may be made without departing from the spirit and scope of this disclosure.

### EXAMPLES

The following examples are put forth so as to provide those of ordinary skill in the art with a complete disclosure and description of how to use the embodiments provided herein and are not intended to limit the scope of the disclosure nor are they intended to represent that the Examples below are all of the experiments or the only experiments performed. Efforts have been made to ensure accuracy with respect to numbers used (e.g. amounts, nutrient combinations, etc.) but some experimental errors and deviations should be accounted for. It should be understood that variations in the methods as described can be made without changing the fundamental aspects that the Examples are meant to illustrate.

### Example 1: Formulation of Product

The individual components (natural compounds), that are present in the compositions disclosed herein, have been individually documented in dozens of studies to be effective and safe in supporting certain immune system and anti-oxidative processes, and the body's natural stem cell physiology, meaning their natural release, circulation and migration to the location that requires repair. However, as individual components, these components have limited activity, and are unable to address in a balanced and comprehensive way the multiple pathways and processes involved in supporting the immune system and other body system functions, stem cell physiology, and overall health, that may be provided by the compositions of this disclosure. The innovative nutrient synergy concept and novel nutrients combination of the compositions of this disclosure In preferred embodiments, the compositions of this disclosure can provide a dietary supplement capable of helping to protect, support, and improve the immune system function and human health.

The composition of this example (StemRCM) was formulated based on analysis of individual compounds and their published research and clinical studies. As presented herein, the antioxidative (ROS), toxicity, anti-aging, lifespan, and health span effects of the composition of this disclosure (i.e., StemRCM) were studied using C. elegans and provided in the following examples. All compounds present in StemRCM are recognized as GRAS and fall under the “food” umbrella as regulated by the Food and Drug Administration (FDA). The StemRCM formula contains a novel, synergistic, and balanced combination of extracts that come from the most potent and fast acting polyphenolic and polysaccharides sources found in nature. In certain embodiments, the present composition provides increased bioavailability and reduced dosage as compared to use of the individual compounds/extracts in the present nutraceutical composition (also referred to herein a blend). The significance of researching the combination of polyphenolic compounds and polysaccharides for health has been recently reported in studies (Scientific Reports, January 2021). The StemRCM formula utilizes the principle of nutrient synergy and multi-targeted heath aim. As shown herein, the compositions of this disclosure allow the user to avoid mega-doses of individual compounds, nutritional imbalances and deficiencies in the body, and can provide much more significant bio-cellular, anti-oxidative, anti-inflammatory and overall health effects versus application of individual compounds provided in high doses, or random combinations of random compounds (British Journal of Nutrition, January 2021).

In certain embodiments, the present nutraceutical composition of this example (StemRCM) comprises an absorption nanotechnology (curcumin extract (Theracurmin®) in the formula for increased anti-oxidative, anti-inflammatory, anti-cancer, cardiovascular, and brain protective health effects. A highly bioavailable curcumin, present in StemRCM, was developed based on innovative nanotechnology that reduces curcumin/turmeric molecules size by 100× times. Curcumin molecules are naturally large and, as such, are not easily absorbed and processed in the body. This unique form of curcumin, present in StemRCM, and decreased size of its molecules, makes this curcumin 27 times faster absorbed after oral administration as compared to the regular curcumin. A clinical study with healthy human volunteers who received orally highly bioavailable curcumin (Theracurmin®) or regular curcumin powder showed that the area under the blood concentration-time curve—AUC of curcumin (Theracurmin®) was 27-fold higher than that of regular curcumin powder (Biol Pharm Bull 2011). These findings demonstrate that curcumin (Theracurmin®) shows a much higher bioavailability than currently available forms and may be useful to exert clinical benefits in humans at a lower dosage.

In certain embodiments of these Examples, the StemRCM formula contains twelve (12) top quality and purity extracts that come from organic and non-GMO natural sources found in nature, such as plants, fruits, and food sources (Table 1). In certain embodiments, the StemRCM formula does not contain any fillers, synthetic preservatives, colorants, and/or additives. The StemRCM formula is provided in quickly absorbed vegetarian capsule. Its absorption time (approximately 20 min) has been tested by the manufacturer of StemRCM (Pharma Natural, Miami Lakes, Fla.).

TABLE 1
Exemplary StemRCM Formulation

| | Mg/capsule | Quantity |
|---|---|---|
| | ‘00’ (measured | (mg/ 3 ‘00’ |
| INGREDIENTS | range) | serving) |
| Beta-glucans (from Oats) | 130 (117-143) | 390 |
| Astragalus (Atragalus | 100 (90-110) | 300 |
| membranaceus) | | |
| Broccoli extract (Brassica | 100 (90-110) | 300 |
| oleracea) | | |
| (Sprouts and seeds extract | | |
| to contain 35 mg | | |
| (10%) Sulforaphane- | | |
| Glucosinolates, | | |
| and supplying Myrosinase | | |
| enzyme) | | |
| Reishi Mushroom | 80 (72-88) | 240 |
| (Ganoderma lucidum) | | |
| Theracurmin ® | 50 (45-55) | 150 |
| (Curcuma longa) | | |
| Developed on innovative | | |
| fast-absorption | | |
| Japanese nanotechnology. | | |
| Trans-Resveratrol 98% | 40 (36-44) | 120 |
| (Polygonum cupsidatum) | | |
| Grape seeds extract | 40 (36-44) | 120 |
| (Vitis rotundifolia) | | |
| Contains Quercitin and | | |
| Ellagic acid. | | |
| Apigenin (Green parsley | 40 (36-44) | 120 |
| extract) | | |
| (Petroselinum crispum) | | |
| EGCG (Camellia sinensis) | 30 (27-33) | 90 |
| (Green tea extract) | | |
| (Standardizes to 85%-95% | | |
| polyphenols, including | | |
| 45% EGCG) | | |
| BioAloe (Aloe vera) | 15 (13.5-16.5) | 45 |
| Vitamin D3 (as Cholecalciferol) | 200 IU | 600 IU |
| | (5 mcg) | (15 mcg) |
| Peppermint (Mentha piperita L) | 10 (9-11) | 30 |
| Total actives: | 637 mg | 1905 mg |
| Vegetable Capsule (‘00’ capsule) | | |

### Example 2: C. elegans Toxicity Assays

This Example describes toxicity assays conducted using Caenorhabditis elegans (C. elegans, or “worm”) as a model organism. StemRCM was tested for lifespan extending activity using a standard C. elegans longevity assay. Initial toxicity and dose finding assays indicated that StemRCM was largely benign and non-toxic as C. elegans tolerated the full range of concentrations with only slight effects. The maximum soluble concentration, however, did increase the variance in growth rate and resulted in decreased survival in a stress assay. Two dosages below the highest showed an encouraging increase in survival in a short-term stress assay and were therefore selected for further study. In a reactive oxygen species (ROS) stress assay, pre-treatment with StemRCM provided better protection against oxidants than the positive control, Vitamin C. The lifespan of worms treated with StemRCM was measured using an established automated lifespan machine to reduce variance and maintain reproducibility. Concurrently, the healthspan of the C. elegans was measured by tracking their activity and morphology for the duration of the lifespan. C. elegans treated with either dosage of StemRCM showed higher levels of activity sustained throughout the lifespan, with the higher dosage showing higher activity. C. elegans treated with the lower dose of StemRCM sustained younger morphological features than the control group over the course of the lifespan. The healthspan and ROS assay data support potential longevity benefits of StemRCM.

A. Materials and Methods

1. C. elegans Maintenance and Media

To prevent chemical modification or metabolism of the test article by the food bacteria, C. elegans were fed on a lawn of inactivated E coli, strain OP50. Cultures of OP50 were inactivated exposure to 0.25% paraformaldehyde for 1 hour followed by 5 washes in M9. Bacteria were dispersed by passing through a 5 μM filter during the wash steps. The quantity and distribution of food bacteria were calibrated to ensure adequate access to food for the duration of assay while maintaining visibility of the C. elegans.

2. Automated Lifespan Machine (ALM)

The ALM used by InVivoBiosystems is based on the C. elegans lifespan machine published by Stroustrup et. al, with proprietary modifications to improve temperature stability and image acquisition. The scanner unit consists of a modified EPSON V850 and images are processed and analyzed using the ALM software (Stroustrup, et al. Nat. Methods, 10, 665-670 (2013)). The machine time-of-death calls are trained and validated using the “storyboarding” feature of the ALM software.

3. Survival Analysis

Time of death calls exported from the ALM software was analyzed and plotted using the Lifelines software package developed by Cam Davidson-Pilon et. al. 11. Additional analysis performed using the OASIS2 analysis software (Seong, et al. Oncotarget, 7: 56147-56152(2016)).

4. Movement and Healthspan Analysis

C. elegans movement was tracked from the images acquired by the ALM during the lifespan assay. C. elegans size and movement features were extracted and analyzed using custom software.

5. Whole Transcriptome Analysis

More than 150 day 1 adult C. elegans per replicate were harvested, cleaned by filtration, and frozen at −80° in Trizol. To extract RNA, samples were thawed, vigorously vortexed, and processed using the Direct-zol RNA Miniprep Kit (Zymo Research). All samples exceeded our threshold for RNA quantity and quality. RNA samples were submitted to Novogene Co. Ltd and subjected to more stringent QC, being tested on a Qubit for concentration and run on an agarose gel and on the Agilent 2100 to assess RNA quality and integrity. All samples had an RNA Integrity Number (RIN) of 8.8 or higher (range is 0-10, with 10 being “perfect”). The total RNA is then enriched for poly-mRNA using oligo(dT) paramagnetic beads. DNA libraries were then constructed from this input mRNA using the NEBNext UltraTM II RNA Library Prep Kit. This creates a ready-to-sequence dsDNA library that retains the strand-specific information in the original mRNA. These libraries were then further tested by the Qubit for concentration and the Agilent 2100 for library size distribution and quality. In order to properly pool the libraries and load them onto sequencing lanes to ensure the correct number of reads per sample, an even more precise quantification of the library was done via qPCR, and the samples were loaded onto the NovaSeq 6000 platform for a paired-end sequencing run of 150 bp for each end (PE150). The loading concentrations were designed to obtain at least 6.0 Gb (which is the number of billion bases of raw data, determined by the number of reads multiplied by the length of each read). Sequencing run data quality control was performed both by Novogene and again internally. The raw data set was analyzed for: 1. The distribution of base quality along the length of the sequencing read. 2. The distribution of error rate along the length of the sequencing read. 3. The distribution of A/T/G/C bases along the length of the sequencing read. 4. The distribution of raw data filtering results based on the following three criteria: a. removing reads containing adapter sequence; b. removing reads with N>10% (where N means “base cannot be determined”); and, c. removing reads with a low quality (Qscore<=5) for 50% or more of its total bases.

B. StemRCM Toxicity Assays

1. Dosage and Toxicity

The ideal dose of a lifespan-active drug will balance providing a high enough dose to be effective with avoiding doses high enough to be either toxic or aversive to the animals. Because C. elegans are physically resistant to environmental chemicals, and worm physiology differs from humans, cultured cells and other animal models, a series of experiments were run to empirically determine an ideal dosage and delivery strategy for a treatment.

2. Solubility and Test Article Delivery Results

The body of C. elegans is encased in a selectively permeable cuticle that only permits some compounds to be absorbed efficiently through the skin, so the most reliable mechanism for delivering compounds to the worms is through ingestion. Water-soluble compounds permeate the media and food and are readily taken up by the worms. Less soluble compounds require a vehicle such as DMSO and work best when combined directly with food. The first step is to check the solubility of the test article and determine the best delivery method.

Solubilization: StemRCM was a heterogeneous mixture that was extracted in equal amounts in both water and DMSO. Insoluble debris were pelleted by centrifugation and the combination of the supernatants from the water and DMSO extraction made the 1× “100 mg/mL” solution.

Delivery strategy: The indicated compound dosage is based on the total volume of the plates with the assumption that the water-soluble compound diffuses throughout the agar. In this case, the 33 mg/mL solution corresponds to a final concentration of 150 μg/mL in solid media. The compound is dissolved in a working solution and then combined directly with the food bacteria before seeding on agar plates. The food spots are dried slowly, allowing the compound to diffuse into the food bacteria and the agar for at least 24 hours before worms are introduced.

3. Growth and Development Assay Results

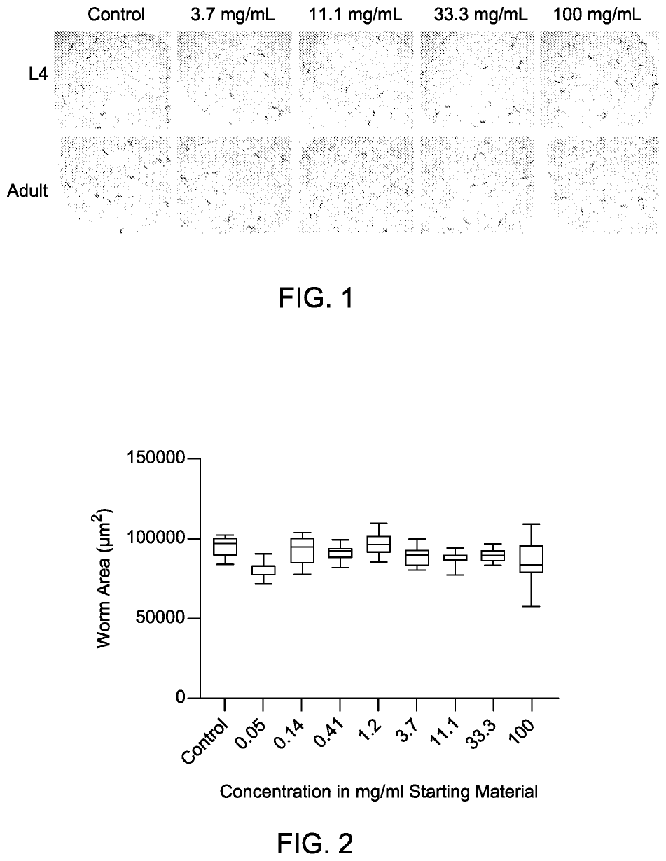

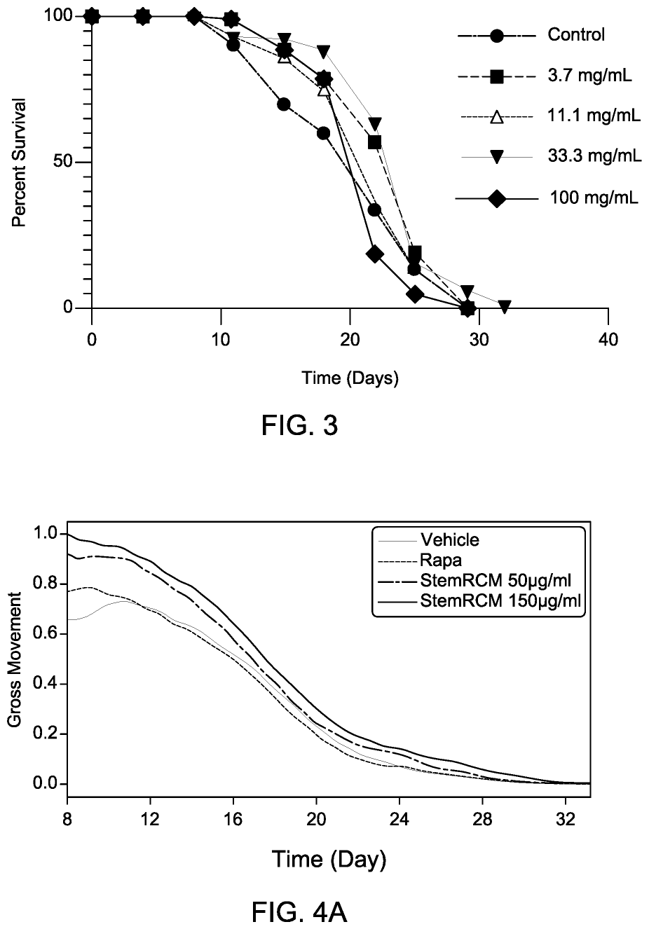

High-resolution imaging and automated detection are used to precisely measure the growth rate of animals from hatching to the first day of adulthood (total of 4 days). The C. elegans growth and development assay is highly sensitive and widely used in toxicology studies. Performing this test over a range of doses helps to identify a set of doses that have a physiological impact and exclude dose ranges that are likely too toxic to benefit lifespan. StemRCM was mostly benign and did not produce any visible changes in growth when larvae were exposed to compound from hatchling to adult (FIGS. 1-3). There was a noticeable increase in variance in the highest dose suggesting that the optimal dose to test effects on longevity would be among lower concentrations.

To measure healthspan, active worms were identified using the ALM scanner images from the lifespan assay. The worms' spatial location on the plate and their morphology were quantified throughout their lifespan to assess healthspan. Worm Activity serves as a proxy for animal health. Changes in spatial distribution of the worms between time points is used to derive gross movement for the population over time. Foreground motion is calculated using an approach called difference based spatial temporal entropy image (DSTEI) (Ma, et al. 2001. “Detecting Motion Object by Spatio-Temporal Entropy.” In IEEE International Conference on Multimedia and Expo, 2001. ICME 2001., 265-68). Worm morphology is measured from the worm contours detected in the images. In the process of aging, worms become shorter and stouter over time and their shape is an indicator of their overall health and biological age. The Length is calculated from the central spline fitted to the worm contour and Width is measured from each worm's widest point. The worms' posture also changes with age as they lose the ability to maintain an elongated position. Average Circularity measures how close the shape and posture comes to being enclosed by a circle. Each of these measures are obtained by averaging data for all active worms detected on a plate, then averaging across different replicate plates of the same condition. All measurements are based on worms that are still alive and moving at the time of quantification. All measures start when worms are placed on the scanner at day 4 of adulthood.

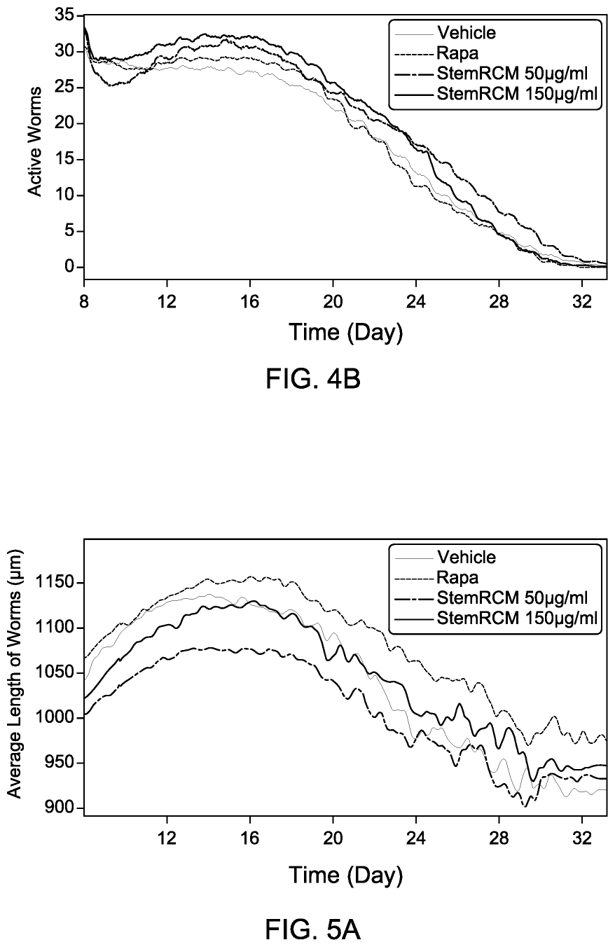

1. Gross Movement. Worm activity was obtained by computationally analyzing the images from the automated lifespan experiment. Worms treated with StemRCM showed greater gross movement from start to end of the lifespan experiment than either vehicle control or Rapamycin-treated (FIGS. 4A-4B). Despite having increased longevity, we have typically observed that Rapamycin treated groups do not show a proportional increase in activity. The high activity of the StemRCM-treated group contrasts with the lifespan data, particularly in how the 150 μg/mL group with the shortened lifespan also showed the highest activity. This raises the question of whether a trade-off might be involved or whether the worms might be stimulated in a way that ends up shortening lifespan.

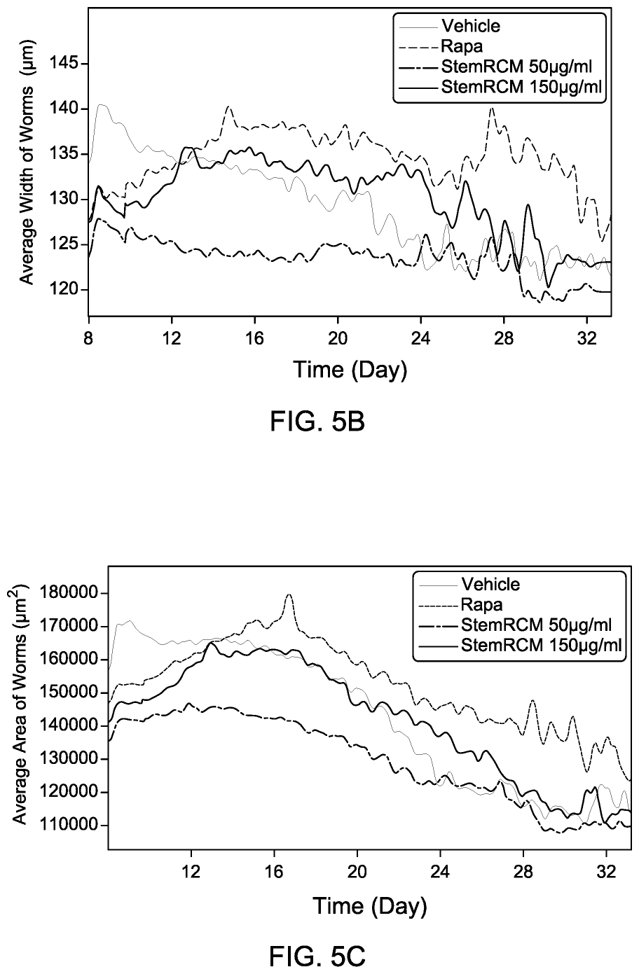

2. Worm Morphology. Worms become shorter, stouter, and smaller as they age so the length and width of the worms can function as an indicator for biological age. Worms treated with 50 μg/mL StemRCM were consistently longer than vehicle control or any of the other conditions, and also had greater width and area (FIGS. 5A-5C). This supports the implication from the lifespan and activity data that this dose is beneficial and that any lifespan increase is not due to starvation, food aversion, or dauer formation. Rapamycin-treated worms, by contrast, tend to be smaller and less active, despite their prolonged lifespan due to the direct action on energy metabolism.

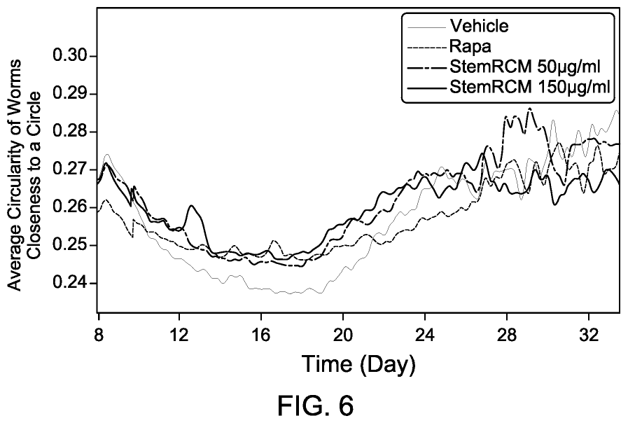

3. Average Circularity. The Average Circularity Assay indicates worm heath by describing how closely the shape and posture of the worms is enclosed within a circle. Healthy, active worms maintain an elongated, albeit sinusoidal posture. As unhealthy and aged worms lose muscle function they increasingly adopt a curled, bunched, or folded state in addition to a stout and wrinkled morphology. Hence, the shape of unhealthy worms is more readily enclosed by a perfect circle—their circularity is closer to 1. Worms treated with either StemRCM or Rapamycin showed slightly greater circularity than the vehicle control group early in the lifespan assay (FIG. 6).

4. Aggregate movement analysis: Animals treated with StemRCM retain a higher level of average movement and activity late into lifespan.

Taken together, the morphology of 50 μg/mL treated worms was consistent with a beneficial effect on healthspan, whereas the higher dose of 150 μg/mL did not show morphological signatures of improved healthspan despite having higher activity.

D. Mechanism of Action Studies

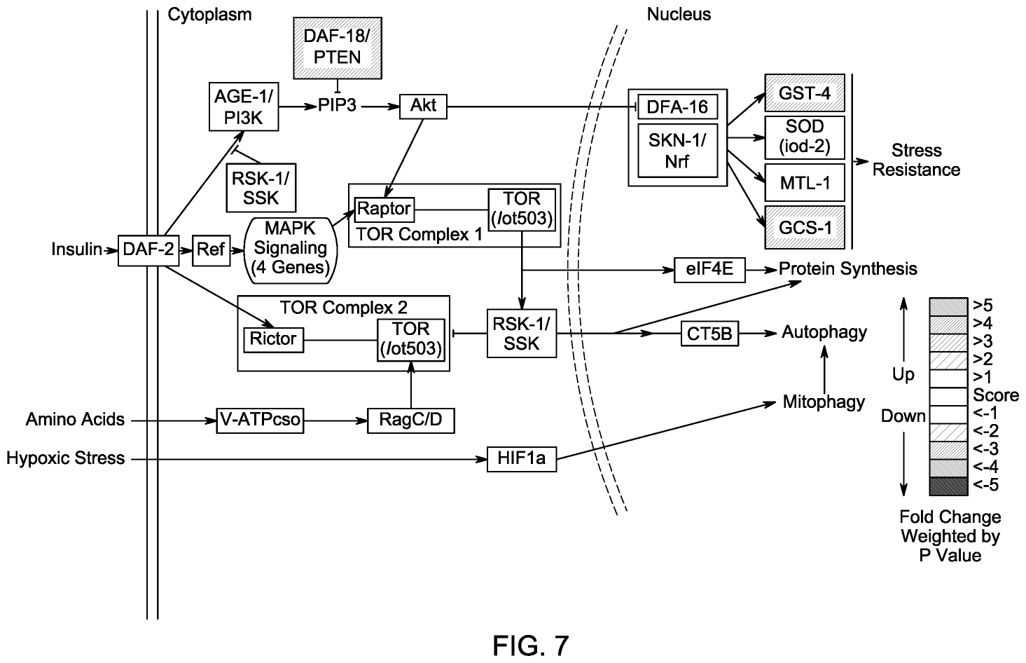

Experiments were conducted to identify which cellular pathways were most likely modulated by treatment with StemRCM and gain insight into how these pathways might contribute to mechanism of action (MoA). To accomplish this, the genes differentially expressed after StemRCM treatment were first mapped to core established longevity pathways from the literature. The mapping was then expanded to intersecting and supporting pathways. This placed the transcriptomic data within the context of well-characterized biological pathways, particularly several related to longevity. Differentially-expressed genes (DEGs) were mapped to known physiological pathways and then examined for coherent linkages between pathways related to longevity. These pathways were drawn from WormBase, KEGG, and other published databases and literature. For each condition, the DEGs were filtered on a more relaxed P value cutoff than the initial differential gene expression to capture broad evidence for a pathway from many small changes as opposed to highly significant individual genes of interest. For visualization, pathway genes were given a score based on the fold change weighted by the log of the P value, and this score was color mapped by magnitude and direction to produce the pathway diagram in FIG. 7. Starting with the canonical longevity pathways, the intersections with supporting pathways were examined to expand the pool of evidence of whether a pathway is impacted by the treatment. Although only a few of the genes might individually have highly significant changes in expression, collectively, the connectivity of the pathways can suggest a coherent hypothesis for a mechanism of action. Since a gene or pathway might be upregulated in response to a stress or downregulated due to relief of the stress, direction of change is not considered, only whether the expression of the genes has changed.

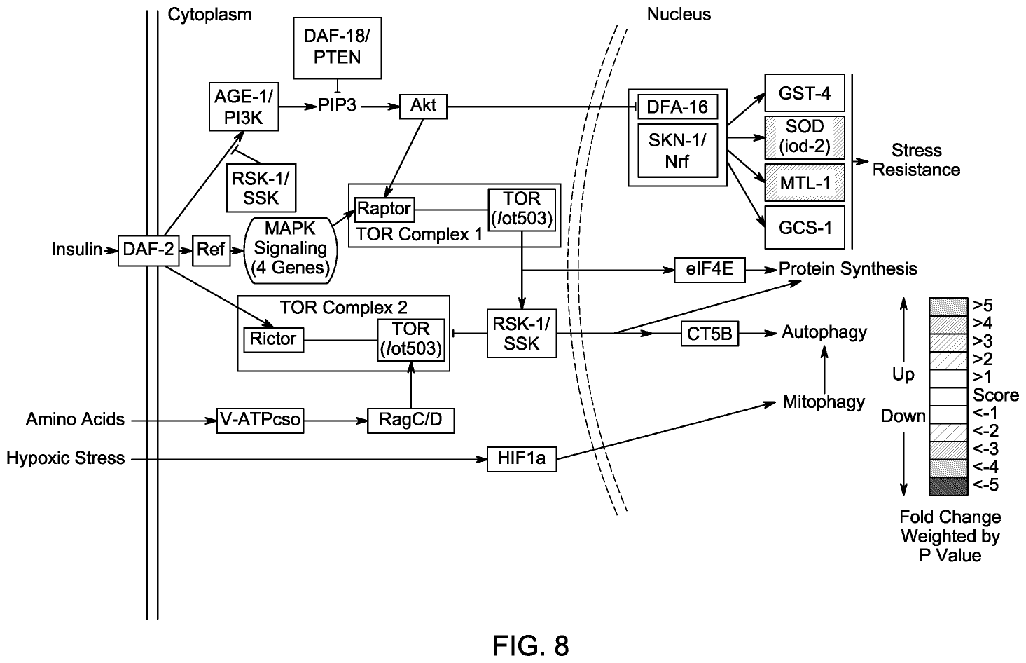

Worms treated with StemRCM showed changes in expression of genes involved with insulin response and energy metabolism, including several key members of canonical longevity pathways (FIGS. 7-8). The Insulin Signaling (IIS) Pathway is most commonly associated with lifespan in part due to its role in caloric restriction. In both young and aged worms, StemRCM treatment altered the expression of key targets of this pathway—mtl-1, sod-2/3, gst-4, and gcs-1—which have all been individually associated with lifespan. Autophagy is another key longevity-associated pathway that is involved in cellular regeneration and homeostasis. One of the top individually upregulated genes was cpr-1, which encodes Cathepsin B, a key enzyme driving autophagy. There were some changes mapped to other longevity pathways such as mitochondrial health, autophagy, and oxidative stress response, but these were less extensive.

Insulin signaling (daf-2, daf-18, rsks-1) in response to StemRCM was also studied. The insulin/insulin-like growth factor-1 signaling (IIS) pathway was the first pathway implicated in genetic regulation of lifespan and aging. The IS signaling pathway regulates longevity through three key components: the worm insulin receptor DAF-2, the kinase AGE-1, and the transcription factor DAF-16. As shown in FIG. 7, StemRCM treatment modulated the expression of several genes linked to this pathway. The discovery that loss of function of the worm insulin receptor DAF-2 could more than double lifespan in C. elegans was a landmark finding that helped launch the field of aging research. In response to StemRCM treatment, daf-18, which encodes the ortholog of human Phosphatase and Tensin (PTEN), was upregulated at Day 3. At Day 10, daf-2 itself was slightly downregulated. Both of these proteins regulate the activity of the Phosphatidylinositol 3-Kinase, AGE-1, which transduces the insulin response signal. The transcriptional output of this pathway is carried out by the transcription factor DAF-16, which controls expression of a large number of genes. Several targets of DAF-16 regulation involved in longevity and stress response, including gst-4 and gcs-1 at Day 3 as well as mtl-1 and sod-2 at Day 10. Each of these four genes has previously been individually associated with lifespan. Their contributions are described below.

Autophagy in response to StemRCM was also studied. Autophagy is a cellular process that catabolizes cellular components to maintain energy homeostasis and protect against stress. Activation of autophagy is associated with increased longevity. The gene cpr-1, which encodes a worm ortholog of Cathepsin B, was significantly downregulated in StemRCM treated worms. Cathepsins control proteloytic degradation within the lysosome. A subset of autophagy, mitophagy promotes longevity through the turnover of declining mitochondria. In addition to cpr-1, several other genes involved with autophagy had less significant changes in expression.

mTOR signaling and energy metabolism (daf-15, rict-1, let-363, rsks-1) in response to StemRCM was also studied. mTOR is a key nutrient sensor and master regulator of growth and energy metabolism in animals. Signaling through mTOR involves two distinct protein complexes, mTORC1 and mTORC2 that regulate different physiological processes. The genes encoding TOR, let-363, and a few other pathway components were slightly downregulated at Day 10. Although TOR signaling pathways share many components and interact with the IS pathway, in this case evidence for strong direct modulation of the TOR pathway was not observed.

Mitochondrial health and oxidative stress (sod-2, oxidative phosphorylation genes) in response to StemRCM was also studied. Mitochondria provide essential energy for the cell. This organelle is also a major source of reactive oxygen species (ROS) which cause oxidative stress and damage to proteins. Disruptions in mitochondria function, particularly in electron transport exacerbates overproduction of ROS. Conditions that promote mitochondrial maintenance and/or turnover (mitophagy) have been linked to extended lifespan and improved health. At Day 10, worms treated with StemRCM showed a slightly increased expression of superoxide dismutase (SOD-2), a key antioxidant enzyme that breaks down reactive oxygen species. Modulation of several genes involved in oxidative phosphorylation by StemRCM treatment can also be an indicator of mitochondrial health and maintenance.

The stress response (mtl-1, skn-1, sod-2) induced by StemRCM was also studied. With compound treatments such as StemRCM, it is common to see expression of many genes involved in response to xenobiotic compounds and innate immune responses that might be responding directly to the drug test compound itself. However, there was also differential expression of key stress response genes linked to longevity: mtl-1, sod-2. The transcription factor SKN-1 is an ortholog of human Nuclear Respiratory Factor (Nrf) that works in conjunction with DAF-16 to activate transcriptional responses to xenobiotic and oxidative stress. Although changes in skn-1 expression itself were not detected, mtl-1 and other genes have been used as markers of SKN-1 protein activity.

Protein translation (ife-2) in response to StemRCM was also studied. Regulation of protein translation in somatic tissues has also been implicated in longevity. The eukaryotic initiation factor 4E (eIF4E) is encoded by the C. elegans gene ife-2. Although there is a connection between TOR signaling and eIF4E activity, knockdown of ife-2 in C. elegans can extend lifespan independently of both IS and TOR signaling, suggesting a possible distinct pathway of life extension.

E. Differential Gene Expression

To identify potential mechanisms of action through which StemRCM could affect aging, global gene expression was analyzed by mRNA sequencing (RNA-Seq). Both young (adult day 3) and aged (adult day 10) worms were collected from the same population of worms tested in the lifespan assay. Three replicates of treated samples were analyzed. Differential gene expression was performed with EdgeR using false likelihood ratio tests based on fitting linear models. The likelihood ratios were used to determine the p-values which were subsequently corrected for using the BH false discovery rate (fdr) method. Ultimately, differentially expressed genes (DEG) were defined as genes with an fdr-corrected p-value of 0.05 or lower, as well as a change in expression of at least 2-fold in a given between-group comparison. In this study, the following comparisons groups were used:

TABLE 3.1
Comparison groups for differential gene
expression

| Group | Experiment | Control |
|---|---|---|
| 1 | StemRCM day 3 | Vehicle day 3 |
| 2 | StemRCM day 10 | Vehicle day 10 |
| 3 | Vehicle day 10 | Vehicle day 3 |
| 4 | StemRCM day 10 | StemRCM day 3 |

TABLE 3.2
StemRCM vs. Vehicle
Control: Top differentially upregulated genes at Day 3.

| | Log | | | |
|---|---|---|---|---|
| Gene | FC | P Value | Protein product | Function description |
| cpr-1 | 0.71 | 0.009 | Cysteine PRotease | cathepsin B-like cysteine protease family |
| | | | related | motifs. |
| ilys-5 | 0.68 | 0.002 | Invertebrate | Is predicted to enable lysozyme activity. |
| | | | LYSozyme | |
| gst-4 | 0.66 | 0.002 | Glutathione S- | Putative glutathione-requiring prostaglandin |
| | | | Transferase | D synthase |
| rpl-39 | 0.62 | 0.010 | Ribosomal Protein, | Large ribosomal subunit L39 protein. |
| | | | Large subunit | |
| Y111B2 | 0.52 | 0.007 | not known | not known |
| A.2 | | | | |
| msra-1 | 0.47 | 0.001 | Methionine | Methionine sulfoxide-S-reductase (MsrA) |
| | | | Sulfoxide Reductase | with experimentally confirmed activity. |
| | | | A | |
| T01C3.3 | 0.44 | 0.000 | not known | Predicted to enable metal ion binding |
| | | | | activity. |
| gst-24 | 0.43 | 0.006 | Glutathione S- | Predicted to enable transferase activity. Is |
| | | | Transferase | involved in innate immune response. |
| | | | | Ortholog of human HPGDS (hematopoietic |
| | | | | prostaglandin D synthase). |
| gst-7 | 0.42 | 0.001 | Glutathione S- | Glutathione S-transferase involved in innate |
| | | | Transferase | immune response |
| mrp-4 | 0.37 | 0.001 | Multidrug | Member of subfamily C of the ATP-binding |
| | | | Resistance Protein | cassette transporters. |
| | | | family | |
| tdc-1 | 0.36 | 0.001 | Tyrosine | Encodes the major C. elegans tyrosine |
| | | | DeCarboxylase | decarboxylase |
| Y51H7C.3 | 0.35 | 0.004 | not known | not known |
| R12C12.7 | 0.35 | 0.002 | not known | not known |
| R04D3.3 | 0.35 | 0.001 | not known | not known |
| B0041.8 | 0.35 | 0.001 | not known | not known |
| oma-2 | 0.34 | 0.002 | Oocyte Maturation | Zinc finger protein of the TIS11 finger type |
| | | | defective | that is paralogous to OMA-1 |
| K08F4.3 | 0.34 | 0.002 | not known | NA |
| Y75B12 | 0.33 | 0.008 | not known | Y75B12B.1 encodes a probable transposase, |
| B.1 | | | | with many C. elegans paralogs and distant |
| | | | | similarity to rotifer and insect transposases. |
| T13F2.2 | 0.33 | 0.009 | not known | NA |
| T24D1.3 | 0.31 | 0.002 | not known | NA |

Top 20 genes differentially expressed under Ergothioneine treatment at Day 3 ranked by P-value. Abridged gene function annotations collected from WormBase are shown; full functional annotations included with data supplement.

TABLE 3.2b
StemRCM vs Vehicle Control: Top differentially downregulate genes at Day 3.

| | Log | | | |
|---|---|---|---|---|
| Gene | FC | P Value | Protein product | Function description |
| F53B2.8 | −1.29 | 1.9E−05 | not known | Affected by several genes including daf-16, |
| | | | | daf-2, and glp-1 based on microarray, tiling |
| | | | | array, RNA-seq, and proteomic studies |
| faah-2 | −1.09 | 5.0E−05 | Fatty Acid Amide | Affected by several genes including daf-16, |
| | | | Hydrolase homolog | daf-2, and rrf-3 based on microarray and |
| | | | | RNA-seq studies An ortholog of human |
| | | | | FAAH (fatty acid amide hydrolase) |
| dod-24 | −0.85 | 1.6E−05 | Downstream Of | Involved in defense response to Gram- |
| | | | DAF-16 (regulated | negative bacterium |
| | | | by DAF-16) | |
| F18C5.10 | −0.82 | 1.5E−03 | not known | Affected by several genes including daf-16; |
| | | | | daf-2; and daf-12 based on tiling array; |
| | | | | microarray; and RNA-seq studies. |
| unc-54 | −0.82 | 9.5E−04 | Un-coordinated | Muscle myosin class II heavy chain (MEW |
| | | | | B); UNC-54 is the major myosin heavy chain |
| | | | | expressed in C. elegans |
| icl-1 | −0.73 | 3.3E−05 | Isocitrate Lyase | Predicted isocitrate lyase/malate synthase, |
| | | | homolog | ICL-1 appears to act downstream of DAF-16 |
| | | | | to influence lifespan. |
| tth-1 | −0.73 | 1.1E−03 | Tetra Thymosin | Encodes a thymosin beta ortholog that |
| | | | (four thymosin | contains four functionally distinct thymosin |
| | | | repeat protein) | beta repeats |
| C06B3.6 | −0.70 | 6.2E−04 | not known | Affected by several genes including daf-2; |
| | | | | eat-2; and sir-2.1 based on microarray and |
| | | | | RNA-seq studies. |
| T19D12.4 | −0.69 | 3.3E−05 | not known | Involved in defense response to Gram- |
| | | | | negative bacterium and innate immune |
| | | | | response. |
| unc-15 | −0.68 | 5.1E−04 | Un-coordinated | Paramyosin ortholog |
| tnt-2 | −0.67 | 9.5E−03 | TropoNin T | Affected by several genes including daf-16; |
| | | | | daf-2; and daf-12 based on microarray; tiling |
| | | | | array; RNA-seq; and proteomic studies. |
| | | | | Ortholog of human TNNT1 (troponin T1, |
| | | | | slow skeletal type). |
| C49G7.10 | −0.66 | 4.9E−03 | not known | Involved in innate immune response. |
| sqst-1 | −0.66 | 1.7E−04 | Sequestosome | Similarity to mammalian sequestosome |
| | | | related | 1(SQSTM1)/p62, a signal transduction or |
| | | | | adaptor protein |
| clec-41 | −0.65 | 1.1E−04 | C-type Lectin | Is predicted to enable carbohydrate binding |
| | | | | activity. Is involved in positive regulation of |
| | | | | chemotaxis. |
| F19B2.5 | −0.65 | 6.2E−03 | not known | Is predicted to enable ATP binding activity |
| | | | | and nucleosome-dependent ATPase activity. |
| F40F8.5 | −0.64 | 8.5E−04 | not known | Is affected by several genes including daf-16; |
| | | | | daf-2; and glp-1 based on microarray; tiling |
| | | | | array; RNA-seq; and proteomic studies. |
| Y58A7A.3 | −0.64 | 1.4E−03 | not known | Affected by several genes including daf-16; |
| | | | | daf-2; and glp-1 based on microarray; tiling |
| | | | | array; and RNA-seq studies. |
| Y94H6A.10 | −0.63 | 7.1E−04 | not known | Affected by several genes including daf-2; |
| | | | | rrf-3; and daf-12 based on proteomic; tiling |
| | | | | array; RNA-seq; and microarray studies. |
| catp-3 | −0.62 | 8.1E−04 | Cation transporting | Predicted to enable ATP binding activity. |
| | | | ATPase | Acts upstream of or within IRE1-mediated |
| | | | | unfolded protein response. Is an ortholog of |
| | | | | human ATP12A (ATPase H+/K+ |
| | | | | transporting non-gastric alpha2 subunit) and |
| | | | | ATP4A (ATPase H+/K+ transporting subunit |
| | | | | alpha). |
| epg-2 | −0.60 | 4.7E−03 | Ectopic P Granules | Involved in macroautophagy and negative |
| | | | | regulation of autophagosome assembly. |

Top 20 genes differentially expressed under Ergothioneine treatment at Day 3 ranked by P-value. Abridged gene function annotations collected from WormBase are shown; full functional annotations included with data supplement.

TABLE 3.3
StemRCM vs. Vehicle Control: Top differentially upregulated genes at Day 10.

| | Log | | | |
|---|---|---|---|---|
| Gene | FC | P Value | Protein product | Function description |
| C50F7.5 | 1.82 | 4.8E−05 | not known | Affected by several genes including daf-16; |
| | | | | daf-2; and glp-1 based on microarray; RNA- |
| | | | | seq; and tiling array studies. |
| asp-1 | 1.25 | 2.0E−11 | Aspartyl Protease | asp-1 encodes a homolog of cathepsin D |
| | | | | aspartic protease; it is transcribed exclusively |
| | | | | in intestinal cells of the late embryo and early |
| | | | | larvae and is not observed in older larvae or |
| | | | | adults; ASP-1 is dispensable for neuronal |
| | | | | degeneration. |
| asp-6 | 1.12 | 9.9E−10 | Aspartyl Protease | aspartic protease. |
| Y53F4B.45 | 0.85 | 4.3E−04 | not known | Affected by several genes including daf-16; |
| | | | | glp-1; and skn-1 based on microarray; tiling |
| | | | | array; and RNA-seq studies. |
| asp-5 | 0.75 | 4.1E−05 | Aspartyl Protease | Is predicted to enable aspartic-type |
| | | | | endopeptidase activity. Is an ortholog of |
| | | | | several human genes including CTSE |
| | | | | (cathepsin E); PGA4 (pepsinogen A4); and |
| | | | | PGC (progastricsin). |
| asp-14 | 0.72 | 4.5E−03 | Aspartyl Protease | asp-14 encodes an aspartyl protease. |
| atp-6 | 0.50 | 4.7E−03 | ATP synthase | The atp-6 gene resides on the mitochondrial |
| | | | subunit | chromosome, and encodes the protein ATP |
| | | | | synthase subunit a; this is the C. elegans |
| | | | | homolog of the MT-ATP6 mitochondrial |
| | | | | membrane ATP synthase (Complex V). |
| ctb-1 | 0.50 | 3.2E−04 | Cytochrome B | The ctb-1 gene resides on the mitochondrial |
| | | | | chromosome, and encodes the cytochrome b |
| | | | | protein of mitochondrial complex III; |
| | | | | mutation of ctb-1 suppresses the slow |
| | | | | embryonic development of isp-1 mutants, |
| | | | | while enhancing their paraquat resistance. |
| act-5 | 0.47 | 1.4E−04 | Actin | act-5 encodes an ortholog of human |
| | | | | cytoplasmic actin. |
| F59B1.2 | 0.46 | 2.2E−03 | not known | Affected by several genes including daf-2; |
| | | | | hsf-1; and elt-2 based on RNA-seq; |
| | | | | microarray; and proteomic studies. |
| ZK813.2 | 0.45 | 9.3E−03 | not known | Is affected by several genes including daf-16; |
| | | | | daf-2; and age-1 based on microarray; RNA- |
| | | | | seq; and tiling array studies. Is affected by |
| | | | | nineteen chemicals including Ethanol; |
| | | | | methylmercuric chloride; and rotenone based |
| | | | | on RNA-seq and microarray studies. |
| C42D4.1 | 0.45 | 2.8E−03 | not known | Affected by several genes including daf-16; |
| | | | | daf-2; and rrf-3 based on microarray; |
| | | | | proteomic; tiling array; and RNA-seq studies. |
| C41G11.1 | 0.45 | 3.0E−04 | not known | Is predicted to enable hydrolase activity. |
| ctc-2 | 0.40 | 9.9E−03 | mitochondrial | The ctc-2 gene resides on the mitochondrial |
| | | | genome encoded | chromosome, and encodes the protein |
| | | | Cytochrome C | cytochrome c oxidase subunit 2; cytochrome |
| | | | oxidase subunit | c oxidase is the component of the respiratory |
| | | | homolog | chain that catalyzes the reduction of oxygen |
| | | | | to water; CTC-2 is one of the 3 subunits (1-3) |
| | | | | that forms the functional core of the enzyme |
| | | | | complex. |
| nduo-5 | 0.37 | 1.3E−03 | mitochondrial | The nduo-5 gene resides on the |
| | | | genome encoded | mitochondria chromosome, and encodes the |
| | | | NADH- (Nadh) | protein NADH-ubiquinone oxidoreductase |
| | | | Ubiquinone | chain 5; this is the C. elegans homolog of the |
| | | | Oxidoreductase | core MT-NDS of mitochondrial |
| | | | chain homolog | NADH: ubiquinone oxidoreductase (Complex |
| | | | | I). |
| col-19 | 0.37 | 3.4E−04 | Collagen | col-19 encodes a member of the collagen |
| | | | | superfamily containing collagen triple helix |
| | | | | repeats (20 copies) that is required for normal |
| | | | | structure of the alae; expressed during the |
| | | | | L2-to-dauer and L4-to-adult molts with |
| | | | | strongest expression in adult animals. |
| nduo-1 | 0.36 | 1.6E−03 | mitochondrial | The nduo-1 gene resides on the |
| | | | genome encoded | mitochondrial chromosome, and encodes the |
| | | | NADH- (Nadh) | protein NADH-ubiquinone oxidoreductase |
| | | | Ubiquinone | chain 1; this is the C. elegans homolog of the |
| | | | Oxidoreductase | core MT-ND1 of mitochondrial |
| | | | chain homolog | NADH: ubiquinone oxidoreductase (Complex |
| | | | | I). |
| iff-1 | 0.36 | 5.5E−04 | Initiation Factor | iff-1 encodes an eIF-5A homolog that affects |
| | | | Five (eIF-5A) | fertility and is required for germ cell |
| | | | homolog | proliferation and for some P granule |
| | | | | components to localize properly; expression |
| | | | | is germline specific and mRNA is expressed |
| | | | | in the distal region of gonads where germ |
| | | | | cells actively proliferate. |
| nduo-4 | 0.36 | 9.6E−03 | mitochondrial | The nduo-4 gene resides on the |
| | | | genome encoded | mitochondrial chromosome, and encodes the |
| | | | NADH- (Nadh) | protein NADH-ubiquinone oxidoreductase |
| | | | Ubiquinone | chain 4; this is the C. elegans homolog of the |
| | | | Oxidoreductase | core MT-ND4 subunit of mitochondrial |
| | | | chain | homolog NADH: ubiquinone oxidoreductase |
| | | | | (Complex I). |
| F14H3.6 | 0.35 | 6.7E−04 | not known | Affected by several genes including daf-16; |
| | | | | daf-2; and rrf-3 based on microarray; tiling |
| | | | | array; and RNA-seq studies. |

Top 20 genes differentially expressed under Ergothioneine treatment at day 10. Abridged gene function annotations collected from WormBase are shown; full functional annotations included with data supplement.

TABLE 3.3b
StemRCM vs. Vehicle Control: Top differentially upregulated genes at Day 10

| | Log | | | |
|---|---|---|---|---|
| gene | FC | P Value | Protein product | Function description |
| ilys-5 | −2.9 | 3.7E−28 | Invertebrate | Is predicted to enable lysozyme activity. |
| | | | Lysozyme | |
| C17H12.8 | −2.5 | 2.1E−58 | not known | Affected by several genes including daf-16; |
| | | | | daf-2; and age-1 based on microarray; RNA- |
| | | | | seq; and proteomic studies. CUB-like domain |
| dod-24 | −2.1 | 2.7E−23 | Downstream Of | Involved in defense response to Gram- |
| | | | DAF-16 (regulated | negative bacterium. |
| | | | by DAF-16) | |
| lipl-5 | −1.8 | 4.6E−19 | Lipase Like | lipl-5 encodes a lipase; lipl-5 is expressed in |
| | | | | the intestine and its expression is negatively |
| | | | | regulated in well-fed animals by MXL-3; |
| | | | | lipl-5 expression is induced upon bacterial |
| | | | | infection. |
| ilys-3 | −1.8 | 6.6E−03 | Invertebrate | Enables lysozyme activity. Is involved in |
| | | | Lysozyme | defense response to Gram-positive bacterium |
| | | | | and determination of adult lifespan. |
| clec-50 | −1.7 | 4.8E−18 | C-type Lectin | Is predicted to enable carbohydrate binding |
| | | | | activity. Is expressed in intestine. |
| lys-7 | −1.6 | 3.9E−09 | Lysozyme | lys-7 encodes an enzyme homologous to an |
| | | | | antimicrobial lysozyme encoded by the |
| | | | | LYS4 gene of the protozoan parasite |
| | | | | Entamoeba histolytica; lys-7 expression is |
| | | | | significantly upregulated in response to |
| | | | | infection with the Gram-negative bacterium |
| | | | | Serratia marcescens; |
| F52E1.14 | −1.5 | 8.8E−24 | not known | Affected by several genes including daf-16; |
| | | | | daf-2; and rrf-3 based on RNA-seq and |
| | | | | microarray studies. |
| vit-3 | −1.5 | 3.4E−05 | Vitellogenin | vit-3 encodes a vitellogenin, a precursor of |
| | | | structural genes | the lipid-binding protein related to vertebrate |
| | | | (yolk protein genes) | vitellogenins and mammalian ApoB-100, a |
| | | | | core LDL particle constituent |
| | | | | (OMIM: 107730); |
| vit-4 | −1.4 | 3.3E−05 | Vitellogenin | Is predicted to enable lipid transporter |
| | | | structural genes | activity and nutrient reservoir activity. |
| | | | (yolk protein genes) | |
| vit-1 | −1.3 | 1.4E−05 | Vitellogenin | Is predicted to enable lipid transporter |
| | | | structural genes | activity and nutrient reservoir activity. |
| | | | (yolk protein genes) | |
| LLC1.2 | −1.3 | 9.2E−08 | not known | Affected by several genes including daf-16; |
| | | | | daf-2; and dpy-10 based on microarray; |
| | | | | proteomic; and RNA-seq studies. |
| cpr-6 | −1.3 | 1.1E−17 | Cysteine Protease | Is predicted to enable cysteine-type peptidase |
| | | | related | activity. Located in lysosome. Human |
| | | | | ortholog(s) of this gene are implicated in |
| | | | | several diseases, including autoimmune |
| | | | | disease of the nervous system (multiple); |
| | | | | carcinoma (multiple); and intracranial |
| | | | | aneurysm. Is an ortholog of human CTSB |
| | | | | (cathepsin B). |
| lys-1 | −1.2 | 1.4E−09 | Lysozyme | lys-1 encodes a putative lysozyme, whose |
| | | | | overexpression increases resistance to |
| | | | | infection by Serratia marcescens. |
| spp-5 | −1.2 | 2.0E−09 | Saposin-like Protein | spp-5 encodes a caenopore, a saposin (B) |
| | | | family | domain-containing protein that is a member |
| | | | | of the saposin-like protein (SAPLIP) |
| | | | | superfamily containing mammalian NK-lysin |
| | | | | and granulysin and the protozoan |
| | | | | amoebapore-like proteins; SPP-5 exhibits |
| | | | | pore-forming activity and functions as an |
| | | | | effector of innate immunity, demonstrating |
| | | | | antimicrobial activity against both Gram- |
| | | | | positive and Gram-negative bacteria. |
| F56C9.7 | −1.1 | 1.2E−13 | not known | F56C9.7 encodes a protein containing a |
| | | | | DUF1261 (Domain of unknown function |
| | | | | 1261) domain that is conserved amongst |
| | | | | nematodes; loss of F56C9.7 activity results |
| | | | | in decreased intestinal dipeptide transport |
| | | | | and slightly increased fat storage; loss of |
| | | | | F56C9.7 activity in a bar-1 mutant |
| | | | | background also results in developmental |
| | | | | variation; large-scale expression studies |
| | | | | indicate that F56C9.7 is expressed in the |
| | | | | intestine. |
| clec-85 | −1.1 | 3.5E−14 | C-type Lectin | Is predicted to enable carbohydrate binding |
| | | | | activity. Is expressed in intestine. |
| asp-13 | −1.1 | 2.6E−07 | Aspartyl Protease | Is predicted to enable aspartic-type |
| | | | | endopeptidase activity. |
| smd-1 | −1.1 | 8.1E−08 | SAM | smd-1 encodes an S-adenosylmethionine |
| | | | Decarboxylase | decarboxylase; SMD-1 functions in |
| | | | | polyamine biosynthesis exhibiting |
| | | | | adenosylmethionine decarboxylase activity |
| | | | | in vitro that is stimulated by putrescine; in |
| | | | | large-scale RNAi screens, loss of smd-1 |
| | | | | results in defective axon guidance and, in a |
| | | | | sensitized genetic background, locomotion |
| | | | | defects. |
| M28.10 | −1.1 | 2.7E−06 | not known | Affected by several genes including daf-2; |
| | | | | rrf-3; and dpy-10 based on microarray and |
| | | | | RNA-seq studies. |

To 20 genes differentially expressed under Ergothioneine treatment at day 10. Abridged gene function annotations collected from WormBase are shown; full functional annotations included with data supplement.

F. Gene Ontology Enrichment

Functional characterization of gene lists using Gene Ontology (GO) enrichment analysis is a common approach in transcriptomic analysis. Once the table of differentially-expressed genes has been created, the annotation of those genes by biological process (BP), molecular function (NF), or cellular compartment (CC), is cataloged and a comparison is made between the likelihood of seeing genes in that category (ontology) being enriched in the list of differentially-expressed genes when compared to a random selection of genes. This allows patterns due to the interactions of multiple genes to emerge. Gene Ontology/Pathway tables presented herein have the following format and definitions:

TABLE 3.4
Top XX differentially-expressed (D.E.) pathways (gene ontology terms)
in treated worms vs control worms on day 3.

| GO ID | Term | Ont | N | Up | Down | P.Up or P.Down |
|---|---|---|---|---|---|---|
| Unique GO | The GO term | Which | Total | # of | # of genes | P-value of gene |
| ID# cataloged | (e.g. immune | ontology | number | genes | sig. down | enrichment (up) |
| at | response, | class | of | sig. up | regulated | or depletion |
| geneontology.org | nucleus, | (BP, MF, | Genes | regulated | in this | (down) in the set |
| | synaptogenes | or CC) | in that | in this | data set | of D.E. genes vs |
| | is) | | GO | data set | | the null set |
| | | | term | | | |

TABLE 3.5
Top under-represented GO terms SternRCM vs control day 3

| GO ID | Term | Ont | N | Up | Down | P value |
|---|---|---|---|---|---|---|
| GO: 0036379 | myofilament | CC | 11 | 0 | 3 | 2.6E−05 |
| GO: 0030017 | sarcomere | CC | 28 | 0 | 3 | 4.8E−04 |
| GO: 0030016 | myofibril | CC | 31 | 0 | 3 | 6.5E−04 |
| GO: 0005865 | striated muscle thin filament | CC | 8 | 0 | 2 | 8.6E−04 |
| GO: 0043292 | contractile fiber | CC | 47 | 0 | 3 | 2.2E−03 |
| GO: 0006955 | immune response | BP | 48 | 0 | 3 | 2.3E−03 |
| GO: 0045087 | innate immune response | BP | 48 | 0 | 3 | 2.3E−03 |
| GO: 0015629 | actin cytoskeleton | CC | 49 | 0 | 3 | 2.5E−03 |
| GO: 0002376 | immune system process | BP | 49 | 0 | 3 | 2.5E−03 |
| GO: 0098857 | membrane microdomain | CC | 19 | 0 | 2 | 5.1E−03 |
| GO: 0045121 | membrane raft | CC | 19 | 0 | 2 | 5.1E−03 |
| GO: 0098589 | membrane region | CC | 19 | 0 | 2 | 5.1E−03 |

GO terms under-represented in StemRCM-treated worms at day 3.
GO ID: Unique GO ID# cataloged at geneontology.org. Full GO term analysis can be found in the data supplement.
Ont: Ontology class biological process (BP), molecular function (MF), cellular compartment (CC)
N: Total number of Genes classified in that GO term.
Up/Down: The number of genes in that GO term (out of N) that are up or down-regulated. Range is shown for all conditions.
P-value: Significance of gene enrichment (up) or depletion (down) in the set of differentially expressed genes vs. the null set.

TABLE 3.6
Top over-represented GO terms StemRCM vs control day 10

| GO ID | Term | Ont | N | Up | Down | P value |
|---|---|---|---|---|---|---|
| GO: 0004190 | aspartic-type endopeptidase | MF | 13 | 3 | 2 | 0.0003 |
| | activity | | | | | |
| GO: 0070001 | aspartic-type peptidase activity | MF | 13 | 3 | 2 | 0.0003 |
| GO: 0140296 | general transcription initiation | MF | 11 | 2 | 1 | 0.0055 |
| | factor binding | | | | | |

GO terms over-represented in StemRCM-treated worms at day 3.
GO ID: Unique GO ID# cataloged at geneontology.org. Full GO term analysis can be found in the data supplement.
Ont: Ontology class biological process (BP), molecular function (MF), cellular compartment (CC)
N: Total number of Genes classified in that GO term.
Up/Down: The number of genes in that GO term (out of N) that are up or down-regulated. Range is shown for all conditions.
P-value: Significance of gene enrichment (up) or depletion (down) in the set of differentially expressed genes vs. the null set.

TABLE 3.7
Top under-represented GO terms StemRCM vs control day 10

| GO ID | Term | Ont | N | Up | Down | P value |
|---|---|---|---|---|---|---|
| GO: 0006952 | defense response | BP | 77 | 0 | 20 | 7.69E−09 |
| GO: 0098542 | defense response to other | BP | 77 | 0 | 20 | 7.69E−09 |
| | organism | | | | | |
| GO: 0044419 | interspecies interaction | BP | 78 | 0 | 20 | 9.82E−09 |
| | between organisms | | | | | |
| GO: 0009607 | response to biotic stimulus | BP | 78 | 0 | 20 | 9.82E−09 |
| GO: 0043207 | response to external biotic | BP | 78 | 0 | 20 | 9.82E−09 |
| | stimulus | | | | | |
| GO: 0051707 | response to other organism | BP | 78 | 0 | 20 | 9.82E−09 |
| GO: 0009605 | response to external | BP | 139 | 0 | 27 | 1.56E−08 |
| | stimulus | | | | | |
| GO: 0006955 | immune response | BP | 48 | 0 | 15 | 4.30E−08 |
| GO: 0045087 | innate immune response | BP | 48 | 0 | 15 | 4.30E−08 |
| GO: 0002376 | immune system process | BP | 49 | 0 | 15 | 5.88E−08 |
| GO: 0005576 | extracellular region | CC | 80 | 2 | 18 | 4.91E−07 |
| GO: 0005581 | collagen trimer | CC | 18 | 1 | 8 | 3.44E−06 |
| GO: 0050830 | defense response to Gram- | BP | 21 | 0 | 8 | 1.37E−05 |
| | positive bacterium | | | | | |
| GO: 0042742 | defense response to | BP | 44 | 0 | 11 | 3.23E−05 |
| | bacterium | | | | | |
| GO: 0009617 | response to bacterium | BP | 44 | 0 | 11 | 3.23E−05 |

GO terms under-represented in StemRCM-treated worms at day 3.
GO ID: Unique GO ID# cataloged at geneontology.org. Full GO term analysis can be found in the data supplement.
Ont: Ontology class biological process (BP), molecular function (MF), cellular compartment (CC)
N: Total number of Genes classified in that GO term.
Up/Down: The number of genes in that GO term (out of N) that are up or down-regulated. Range is shown for all conditions.
P-value: Significance of gene enrichment (up) or depletion (down) in the set of differentially expressed genes vs. the null set.

G. Summary of Results

Dosage and toxicity. StemRCM was a heterogeneous mixture that was extracted into both water and DMSO to capture all available solutes. StemRCM was delivered by mixing maximal water and DMSO fractions directly with food and seeding onto solid media. The growth assay showed the maximum non-toxic dose for StemRCM to be 33.3 mg/mL solution. The acute toxicity assay showed the maximum non-toxic dose for StemRCM to be 33.3 mg/mL solution. The optimal dose ranges used in these studies were 33.3 mg/mL or approximately 150 μL final concentration in solid media plus one lower dose of 11.1 mg/mL or 50 μm/mL final.

Reactive oxygen species assay. Pre-treatment with StemRCM provided greater protection against a potent oxidant, Paraquat, than the positive control, Vitamin C.

Oxidative stress has been implicated in the pathogenesis of many diseases and proposed to be one of the main causes of aging. Like mammals, the nematode C. elegans has well-defined stress defense systems for protection from toxic compounds (Van Raamsdonk and Hekimi 2010) and serves as sensitive testing model for screening sensitivity to ROS, oxidative stress recovery, toxicity and other effects of pharmaceutical drugs and nutraceuticals.

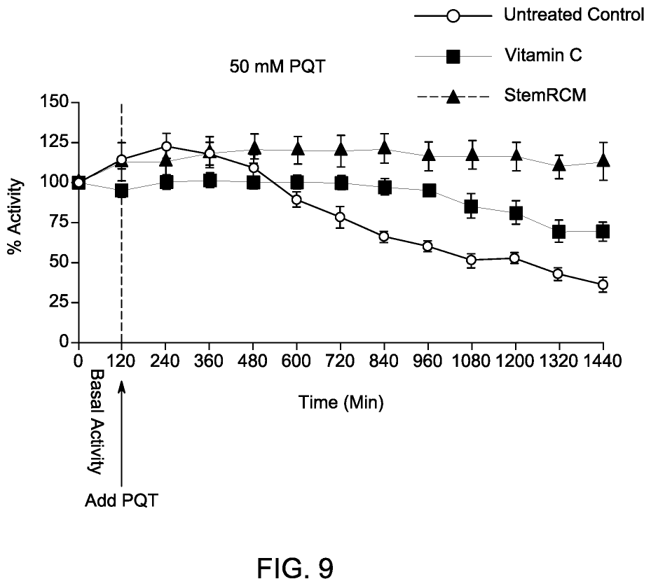

In the studies presented here, the sensitivity to oxidative stress (ROS) and toxicity has been tested by measuring the percentage of C. elegans movement in time intervals—up to 24 hrs using 10 mM and 50 mM paraquat treatment for StemRCM testing group, control group, and group treated with vitamin C. In StemRCM group an increased and lasting movement/vitality of C. elegans, resistance to paraquat/toxicity and oxidative stress has been observed at both concentrations 10 mM and 50 m, and has increased with time of exposure. A significant and increased oxidative stress resistance in StemRCM group has been observed with 50 mM pre-treatment after 2 hrs exposure to the synergistic blend, and continued up to 24 hrs compared to the untreated control ((p=<0.0001), and vitamin C group. Moreover, 50 mM paraquat exposure has been totally inhibited by the StemRCM. These results may indicate and confirm the superiority of synergy of polyphenolic compounds and polysaccharides in StemRCM in reducing detrimental cellular effects of ROS versus a single compound—vitamin C (FIG. 9).

The data presented herein demonstrate the significant potential of the synergistic and innovative blend of polyphenolic compounds and polysaccharides, present in StemRCM, on the improvement of longevity pathways, including mitochondria, healthspan and lifespan, significant reduction of oxidative stress, and the support of detoxification pathways. The synergistic blend in StemRCM modulated several genes, genes responses, and lifespan related pathways, such as the Insulin Signaling (IIS), mtl-1, sod-2/3, gst-4, and gcs-1—which have all been individually associated with lifespan, the transcription factor DAF-16, which controls expression of a large number of genes, and genes involved in oxidative phosphorylation. Modulation of several genes involved in oxidative phosphorylation by StemRCM treatment can also be an indicator of mitochondrial health and maintenance. For instance, mitochondria dysfunction has been reported in studies to trigger cancer, heart diseases, and other diseases.

The discovery that loss of function of the worm insulin receptor DAF-2 could more than double lifespan in C. elegans was a landmark finding that helped launch the field of aging research. Since a gene or pathway might be upregulated in response to a stress or downregulated due to relief of the stress, the direction of change is not considered, only whether the expression of the genes has changed. Worms treated with StemRCM showed changes in expression of genes involved with insulin response and energy metabolism, including several key members of canonical longevity pathways (FIGS. 7-8). The Insulin Signaling (IIS) Pathway is most commonly associated with lifespan in part due to its role in caloric restriction. In both young and aged worms, StemRCM treatment altered the expression of key targets of this pathway—mtl-1, sod-2/3, gst-4, and gcs-1—which have all been individually associated with lifespan. Autophagy is another key longevity-associated pathway that is involved in cellular regeneration and homeostasis. One of the top individually upregulated genes was cpr-1, which encodes Cathepsin B, a key enzyme driving autophagy. There were some changes mapped to other longevity pathways such as mitochondrial health, autophagy, and oxidative stress response, but these were less extensive. Here we summarize how these pathways might contribute to longevity within the context of the StemRCM gene expression data.

These studies aimed to identify which cellular pathways were most likely modulated by treatment with StemRCM and gain insight into how these pathways might contribute to mechanism of action (MoA). To accomplish this, we first mapped the genes differentially expressed after StemRCM treatment to core established longevity pathways from the literature. Then we expanded the mapping to intersecting and supporting pathways. This placed the transcriptomic data within the context of well-characterized biological pathways, particularly several related to longevity. In response to StemRCM treatment, daf-18, which encodes the ortholog of human Phosphatase and Tensin (PTEN), was upregulated at Day 3. At Day 10, daf-2 itself was slightly downregulated. Both of these proteins regulate the activity of the Phosphatidylinositol 3-Kinase, AGE-1, which transduces the insulin response signal. The transcriptional output of this pathway is carried out by the transcription factor DAF-16, which controls expression of a large number of genes15. Several targets of DAF-16 regulation involved in longevity and stress response, including gst-4 and gcs-1 at Day 3 as well as mtl-1 and sod-2 at Day 10. Each of these four genes has previously been individually associated with lifespan. Activation of autophagy is associated with increased longevity. The gene cpr-1, which encodes a worm ortholog of Cathepsin B, was significantly downregulated in StemRCM treated worms. Cathepsins control proteolytic degradation within the lysosome. A subset of autophagy, mitophagy, promotes longevity through the turnover of declining mitochondria. In addition to cpr-1, several other genes involved with autophagy had less significant changes in expression.

### Example 3: The Synergistic Effects of the Compounds in the Product on Human Immune Cells and the Immune System

In an exemplary embodiment, the composition of this disclosure may include the following properties and relationship between the components. The listed all-natural and organic compounds may help to increase each other's antioxidant and natural immunomodulatory responses and effects through various bio-cellular and genetic pathways.

While certain embodiments have been described in terms of the preferred embodiments, it is understood that variations and modifications will occur to those skilled in the art. Therefore, it is intended that the appended claims cover all such equivalent variations that come within the scope of the following claims

1. A composition comprising an admixture of nutraceutical components selected from beta-glucans from oats (Avena sativa); Astragalus extract; Broccoli sprouts extract (Brassica oleracea); Ganoderma lucidum (Reishi Mushroom) extract; Curcuma longa extract; Trans-Resveratrol from Polygonum cupsidatum; grape seed extract from Vitis rotundifolia; Apigenin from Green parsley extract (Petroselinum crispum), green tea extract comprising epigallocatechin-3-gallate (EGCG) from Camellia sinensis; Aloe vera extract; peppermint extract from Mentha piperita L; and, Vitamin D3 (Cholecaliferol).

2. The composition of claim 1 comprising 110-150 mg of beta-glucans from oats; about 80-120 mg of Astragalus extract; about 80-120 mg of Brassica oleracea extract; about 70-90 mg of Ganoderma lucidum (Reishi Mushroom) extract; about 40-60 mg of Curcuma longa extract; about 30-50 mg of Trans-Resveratrol from Polygonum cupsidatum; about 30-50 mg of grape seed extract from Vitis rotundifolia; about 30-50 mg of apigenin from Green parsley extract (Petroselinum crispum), about 20-40 mg of green tea extract comprising epigallocatechin-3-gallate (EGCG) from Camellia sinensis; about 10-20 mg of Aloe vera extract; about 5-15 mg peppermint extract from Mentha piperita L; and, about 150-250 IU or 1-5 microgram of Vitamin D3 (Cholecaliferol).

3. The composition of claim 1 comprising about 117-143 mg of the beta-glucans; about 80-120 mg of the Astragalus extract; about 90-110 mg of the Brassica oleracea extract; about 80 mg of the Ganoderma lucidum (Reishi Mushroom) extract; about 45-55 mg of the Curcuma longa extract; about 36-44 mg of the Trans-Resveratrol; about 36-44 mg of the grape seed extract; about 36-44 mg of the green parsley extract; about 27-33 mg of the green tea extract; about 13.5-16.5 mg of the Aloe vera extract; about 9-11 mg of the peppermint extract; and, about 1.8-3.0 micrograms of the vitamin D3 (Cholecaliferol).

4. The composition of claim 2 comprising about 130 mg of the beta-glucans; about 100 mg of the Astragalus extract; about 100 mg of the Brassica oleracea extract; about 80 mg of the Ganoderma lucidum (Reishi Mushroom) extract; about 50 mg of the Curcuma longa extract; about 40 mg of the Trans-Resveratrol; about 40 mg of the grape seed extract; about 40 mg of the green parsley extract; about 30 mg of the green tea extract; about 15 mg of the Aloe vera extract; about 10 mg of the peppermint extract; and, about 2 micrograms or 200 IU of the vitamin D3 (Cholecaliferol).

5. The composition of claim 1 further comprising a pharmaceutically acceptable excipient.

6. The composition of claim 5 suitable for intravenous (IV) administration.

7. The composition of claim 1 contained in a capsule.

8. The composition of claim 7 wherein the capsule is a vegetarian capsule.

9. The composition of claim 7, wherein the capsule comprises a total of 500-700 mg, optionally about 640 mg, of the nutraceutical components.

10. A dosage form comprising one or more capsules comprising 1500-2100 mg, optionally about 1900 mg, of the nutraceutical components of claim 1.

11. (canceled)

12. A method for altering the expression of one or more genes in a mammal, the one or more genes being a mammalian homologue selected from the group consisting of a gene in the C. elegans Insulin Signaling Pathway, mtl-1, sod-2/3, gst-4, gcs-1, cpr-1, daf-18, and daf-2; the method comprising administering a composition to a mammal, wherein the composition comprises an admixture of at least one polyphenolic compound and at least one polysaccharide compound, wherein the compounds are selected from beta-glucans from oats (Avena sativa); Astragalus extract; Broccoli sprouts extract (Brassica oleracea); Ganoderma lucidum (Reishi Mushroom) extract; Curcuma longa extract; Trans-Resveratrol from Polygonum cupsidatum; grape seed extract from Vitis rotundifolia; Apigenin from Green parsley extract (Petroselinum crispum), green tea extract comprising epigallocatechin-3-gallate (EGCG) from Camellia sinensis; Aloe vera extract; peppermint extract from Mentha piperita L; and, Vitamin D3 (Cholecaliferol).

13. Use of the composition of claim 1 as an anti-aging treatment comprising administering the composition to a mammal, the composition comprises an admixture of at least one polyphenolic compound and at least one polysaccharide compound, wherein the compounds are selected from beta-glucans from oats (Avena sativa); Astragalus extract; Broccoli sprouts extract (Brassica oleracea): Ganoderma lucidum (Reishi Mushroom) extract; Curcuma longa extract; Trans-Resveratrol from Polygonum cupsidatum; grape seed extract from Vitis rotundifolia; Apigenin from Green parsley extract (Petroselinum crispum), green tea extract comprising epigallocatechin-3-gallate (EGCG) from Camellia sinensis; Aloe vera extract; peppermint extract from Mentha piperita L; and, Vitamin D3 (Cholecaliferol).

14. Use of the composition of claim 1 as a dietary supplement.

15. (canceled)

16. The use of claim 14, wherein the nutraceutical composition supplements the immune system.

17. The use of claim 14, wherein the nutraceutical composition reducing in vivo inflammation.

18. The use of claim 14, wherein the nutraceutical composition improves bioavailability of each nutraceutical component as compared to each individual nutraceutical component administered alone.

19. A composition comprising an admixture of at least one polyphenolic compound and at least one polysaccharide compound, wherein the compounds are selected from beta-glucans from oats (Avena sativa); Astragalus extract; Broccoli sprouts extract (Brassica oleracea); Ganoderma lucidum (Reishi Mushroom) extract; Curcuma longa extract; Trans-Resveratrol from Polygonum cupsidatum; grape seed extract from Vitis rotundifolia; Apigenin from Green parsley extract (Petroselinum crispum), green tea extract comprising epigallocatechin-3-gallate (EGCG) from Camellia sinensis; Aloe vera extract; peppermint extract from Mentha piperita L.

20. (canceled)

21. The use of claim 20 as a treatment in an organism for exposure to oxidant chemicals and/or oxidative stress comprising administering the composition or dosage form to the organism.

22. The use of claim 21, wherein the organism is a mammal.

23. The use of claim 21, wherein the organism is a human.

24-26. (canceled)
